(19) United States
(12) Patent Application Publication
(10) Pub. No.: US 2023/0038666 A1
(43) Pub. Date: Feb. 9, 2023

(54) NANOBRIDGE BIOSENSOR AND MEMORY ARRAY

(71) Applicant: Roswell Biotechnologies, Inc., San Diego, CA (US)

(72) Inventors: Sungho Jin, San Diego, CA (US); Chulmin Choi, San Diego, CA (US); Jihye Baek, San Diego, CA (US); Paul W. Mola, San Diego, CA (US); Barry Merriman, San Diego, CA (US)

(73) Assignee: Roswell Biotechnologies, Inc., San Diego, CA (US)

(21) Appl. No.: 17/789,512

(22) PCT Filed: Dec. 28, 2020
(86) PCT No.: PCT/US2020/067187

§ 371 Date: Jun. 27, 2022

Related U.S. Application Data
(63) Continuation of application No. 16/840,755, filed on Apr. 6, 2020.
(60) Provisional application No. 62/954,272, filed on Dec. 27, 2019.
(60) Provisional application No. 62/954,306, filed on Dec. 27, 2019.
(60) Provisional application No. 62/954,324, filed on Dec. 27, 2019.
(60) Provisional application No. 62/954,292, filed on Dec. 27, 2019.
(60) Provisional application No. 62/830,231, filed on Apr. 5, 2019.

(51) Int. Cl. G01N 33/487; G01N 27/327; C12Q 1/6869; C12Q 1/48

(52) U.S. Cl. CPC G01N 33/48721; G01N 27/3271; C12Q 1/6869; C12Q 1/485; G01N 27/3275

(57) ABSTRACT

Various aspects of the present disclosure provide methods, apparatus and systems for single-molecule biosensors having nanowire or nanoribbon bridges between electrodes for sequencing and information storage and reading. In various embodiments, the present disclosure provides nanofabrication of biomolecular sensing devices beginning with parallel arrangements of transferable nanowires or nanoribbons, and provides in general methods of manufacturing biosensor devices for sequencing DNA or RNA and analyzing biomolecules.

### CROSS-REFERENCE TO RELATED APPLICATIONS

This application is a National Phase of and claims priority to, and the benefit of, International Application No. PCT/US2020/067187 entitled “Method, Apparatus and System for Single-Molecule Polymerase Biosensor with Transition Metal Nanobridge” filed Dec. 28, 2020, which claims priority to, and the benefit of, U.S. Non-Provisional patent application Ser. No. 16/840,755 filed Apr. 6, 2020, entitled “Method, Apparatus and System for Single-Molecule Polymerase Biosensor with Transition Metal Nanobridge”; U.S. Provisional Patent Application Ser. No. 62/954,272 filed Dec. 27, 2019, entitled “Single-Molecule Polymerase Biosensor Comprising Transition Metal Dichalcogenide Nanobridge for Sequencing, Information Storage and Reading”; U.S. Provisional Patent Application Ser. No. 62/954,306 filed Dec. 27, 2019, entitled “Groove and Step Pre-Aligned and Transferred Nano-Bridge Biosensor and Memory Array, and Method and Uses Thereof”; U.S. Provisional Patent Application Ser. No. 62/954,324 filed Dec. 27, 2019, entitled “Shear-Aligned and Transferred Nano-Bridge Biosensor and Memory Devices, and Method and Uses Thereof”; and U.S. Provisional Patent Application Ser. No. 62/954,292 filed Dec. 27, 2019, entitled “Pre-Aligned and Transferred, SOI Based Nano-Bridge Biosensor Array and Memory, Comprising Single Biomolecule Sensor, and Method and Uses Thereof” These disclosures are incorporated herein by reference in their entireties for all purposes.

### FIELD

The disclosure relates to biosensors, and in particular to methods, apparatus and systems for single-molecule biosensors having a transition metal dichalcogenide or silicon nanowire or nanoribbon bridge for sequencing, information storage and reading.

### BACKGROUND

Analysis of biomolecules including DNAs and genomes has received an increasing amount of attention in recent years in various fields of precision medicine or nanotechnology. The seminal work of Maclyn McCarty and Oswald T. Avery in 1946 (see, “Studies On The Chemical Nature Of The Substance Inducing Transformation Of Pneumococcal Types II. Effect Of Desoxyribonuclease On The Biological Activity Of The Transforming Substance,” The Journal of Experimental Medicine 83(2), 89-96 (1946)), demonstrated that DNA was the material that determined traits of an organism. The molecular structure of DNA was then first described by James D. Watson and Francis HC Crick in 1953 (see a published article, “Molecular structure of nucleic acids.”, Nature 171,737-738 (1953)), for which they received the 1962 Nobel Prize in Medicine. This work made it clear that the sequence of chemical letters (bases) of the DNA molecules encode the fundamental biological information. Since this discovery, there has been a concerted effort to develop means to actually experimentally measure this sequence. The first method for systematically sequencing DNA was introduced by Sanger, et al in 1978, for which he received the 1980 Nobel Prize in Chemistry. See an article, Sanger, Frederick, et al., “The nucleotide sequence of bacteriophage φX174.” Journal of Molecular Biology 125, 225-246 (1978).

Sequencing techniques for genome analysis evolved into utilizing automated commercial instrument platform in the late 1980's, which ultimately enabled the sequencing of the first human genome in 2001. This was the result of a massive public and private effort taking over a decade, at a cost of billions of dollars, and relying on the output of thousands of dedicated DNA sequencing instruments. The success of this effort motivated the development of a number of “massively parallel” sequencing platforms with the goal of dramatically reducing the cost and time required to sequence a human genome. Such massively parallel sequencing platforms generally rely on processing millions to billions of sequencing reactions at the same time in highly miniaturized microfluidic formats. The first of these was invented and commercialized by Jonathan M. Rothberg's group in 2005 as the 454 platform, which achieved thousand fold reductions in cost and instrument time. See, an article by Marcel Margulies, et al., “Genome Sequencing in Open Microfabricated High Density Picoliter Reactors,” Nature 437, 376-380 (2005). However, the 454 platform still required approximately a million dollars and took over a month to sequence a genome.

The 454 platform was followed by a variety of other related techniques and commercial platforms. See, articles by M. L. Metzker, “Sequencing Technologies—the Next Generation,” Nature Reviews Genetics 11(1), 31-46 (2010), and by C. W. Fuller et. al, “The Challenges of Sequencing by Synthesis,” Nature Biotechnology 27(11), 1013-1023 (2009). This progress lead to the realization of the long-sought “$1,000 genome” in 2014, in which the cost of sequencing a human genome at a service lab was reduced to approximately $1,000, and could be performed in several days. However, the highly sophisticated instrument for this sequencing cost nearly one million dollars, and the data was in the form of billions of short reads of approximately 100 bases in length. The billions of short reads often further contained errors so the data required interpretation relative to a standard reference genome with each base being sequenced multiple times to assess a new individual genome.

Thus, further improvements in quality and accuracy of sequencing, as well as reductions in cost and time are still needed. This is especially true to make genome sequencing practical for widespread use in precision medicine (see the aforementioned article by Fuller et al), where it is desirable to sequence the genomes of millions of individuals with a clinical grade of quality.

While many DNA sequencing techniques utilize optical means with fluorescence reporters, such methods can be cumbersome, slow in detection speed, and difficult to mass produce to further reduce costs. Label-free DNA or genome sequencing approaches provide advantages of not having to use fluorescent type labeling processes and associated optical systems, especially when combined with electronic signal detection that can be achieved rapidly and in an inexpensive way.

In this regard, certain types of molecular electronic devices can detect single molecule, biomolecular analytes such as DNAs, RNAs, proteins, and nucleotides by measuring electronic signal changes when the analyte molecule is attached to a circuit, for example, a field effect transistor device. Such methods are label-free and thus avoid using complicated, bulky and expensive fluorescent type labeling apparatus. These methods can be useful for lower cost sequencing analysis of DNA, RNA and genome.

Certain types of molecular electronic devices can detect the biomolecular analytes such as DNAs, RNAs, proteins, and nucleotides by measuring electronic signal changes when the analyte molecule is attached to the circuit comprising a pair of conductive electrodes. Such methods are label-free and thus avoids using complicated, bulky and expensive fluorescent type labeling apparatus. One example of sequencing-by-synthesis approach is to utilize a single molecule polymerase with incorporated DNAs, the sequence of which is detected through a current pulse signal when each type of the nucleotides (A,T,C,G) is attached to the polymerase complex with a distinct electrical signal.

While current molecular electronic devices can electronically measure molecules for various applications, they lack the reproducibility as well as scalability and manufacturability needed for rapidly sensing many analytes at a scale of up to millions in a practical manner. Such highly scalable methods are particularly important for DNA sequencing applications, which often need to analyze millions to billions of independent DNA molecules. In addition, the manufacture of current molecular electronic devices is generally costly due to the high level of precision needed.

### SUMMARY

Disclosed herein are principles that provide new and improved sequencing apparatuses, device structures and methods using two-dimensional layer structured semiconductors, which can provide reliable DNA genome analysis performance and are amenable to scalable manufacturing. In various embodiments, the present disclosure provides nanofabrication of biomolecular sensing devices and fabrication of devices for analyzing DNA and related biomolecules. In various embodiments, the present disclosure provides DNA-based memory systems.

In various embodiments herein, biomolecular sensors comprise a nanobridge structure disposed over a nanogap between electrodes in a pair of electrodes, wherein the nanobridge comprises a transition metal dichalcogenide (TMD) material, a silicon material, e.g., pure crystalline silicon or various doped silicon semiconductor materials, a carbon nanotube, graphene or various semiconductors.

Two dimensional (2D) layered transition metal dichalcogenides (TMDs) materials and devices have attracted a great deal of interest due to their novel electronic, physical and chemical characteristics. One example is MoS₂ which can be incorporated as a sensor device. MoS₂ type 2D materials can be a single layered material or several layered material, and can be obtained by various techniques, such as e.g., by isolation of very thin MoS₂ layer through mechanical exfoliation, physical or chemical vapor deposition, molecular beam epitaxy (MBE) type construction or sulfurization of transition metal layer such as Mo or W.

Transition metal dichalcogenide (TMD) monolayers are in general atomically thin semiconductors of the type MX₂, which M a transition metal atom (notably Mo, W, or Ti, Zr, Hf, V, Nb, Ta, Tc, Re, Co, Rh, Ir, Ni, Pd, Pt) and X a chalcogen atom (S, Se, or Te). One layer of M atoms is sandwiched between two layers of X atoms. A MoS₂ monolayer can be about 6.5 Å thick. TMD monolayers of e.g., MoS₂, WS₂, MoSe₂, WSe₂, MoTe₂ have a direct band gap, and can be used in electronics as transistors or sensors. Either monolayer TMD or few layer TMD can be structurally modified, to be utilized as solid state DNA or genome sensors. Being an ultrathin direct bandgap semiconductor, a transition metal dichalcogenide such as single-layer MoS₂ may have potential for widespread applications in nanoelectronics, optoelectronics, and energy harvesting.

The layered TMDs typically have a hexagonal type structure with space group P63/mmc. It should be noted that monolayers of TMD materials are not just one atom thick as graphene, but are made up with tri-atomic thick layers consisting of metal atoms (such as Mo or W) sandwiched between two layers of chalcogen atoms (such as S, Se, or Te). The atoms in-plane in MoS₂ type 2D materials are put together and bonded by strong covalent bonds. The adjacent layers of TMD like MoS₂ along the thickness direction are joined together by a weak van der Wall force binding. This force is strong enough to hold the layers together with mechanical integrity. The TMD materials provide interesting and unique possibilities to design electronic devices involving hetero structures. The direct band gap of TMD monolayers is tunable with the application of the mechanical strain.

Single nucleotide identification and DNA sequencing have already been demonstrated with biological nanopores or solid state nanopores such as those in graphene and MoS₂ layers. A DNA type molecule is threaded through a nanopore under an applied electric field, so that the sequence of nucleotides is read by monitoring small changes in the ionic current flowing through the pore, which are induced by individual nucleotides temporarily residing within the pore during threading. However, the fragility of such pores, together with difficulties related to reproducible and low noise measurement of detection signals in nanopore sequencing methods in general are some of the current issues that need to be addressed.

The disclosed principles provide, among others, new biomolecular sensor devices and associated methods, employing transition metal dichalcogenide nanoribbons as a component of molecular bridge, which in turn comprises an attached, preferably single molecule polymerase to analyze DNA lines or fragments by step by step attachments of nucleotides or short DNA fragments.

Various embodiments are disclosed herein regarding specially processed, 2D layer-containing enzyme polymerase sensor device structures and methods of manufacture for a multitude of devices for use in electronic DNA, RNA or genome sequencing systems. Unique geometrical modifications are made so as to enable a construction of sensor device comprising only a single molecule polymerase enzyme for more accurate electronic analysis. Such label-free, single molecule based sequencing analysis systems utilize preferably a nanoscale dimension-controlled, transition metal dichalcogenide (TMD) micro-ribbon or nano-ribbon bridge. The electronic system may also be used in analyzing other types of biomolecules, such as proteins, depending on how the molecular sensors are functionalized to interact with biomolecule sensing targets. The TMD-based sequencing systems disclosed here can be assembled into a massively parallel configuration for rapid analysis of targets including nucleotides, in particular for applications to sequencing of a DNA molecule, or a collection of such molecules constituting an entire human genome. Such systems in the present disclosure can also be used for DNA-based information storage, for example, for archival storage of huge volume of information in human society.

In various embodiments of the present disclosure, a method of manufacturing a sensor device comprises: forming a plurality of parallel aligned nanowires or nanoribbons on a substrate; providing a device structure comprising pairs of electrodes disposed in a parallel array on a surface of the device structure, each pair of electrodes in the array comprising a first electrode and a second electrode spaced apart from the first electrode by a nanogap; transferring a group of the parallel aligned nanowires or nanoribbons from the substrate onto the array of pairs of electrodes such one nanowire or nanoribbon electrically connects the first and second electrodes in each pair of electrodes and forms a bridge suspended over the nanogap of each electrode pair; patterning a dielectric layer over the parallel aligned nanowires or nanoribbons so as to leave openings in the dielectric layer, wherein each opening exposes a single region of nanowire or nanoribbon disposed over each nanogap in each pair of electrodes; and attaching a molecule to each exposed region of nanowire or nanoribbon.

In various embodiments, the molecule comprises a DNA polymerase enzyme or an RNA polymerase enzyme.

In various embodiments, the forming comprises growing the nanowires or nanoribbons on parallel steps or within parallel grooves configured in the substrate.

In various embodiments, the forming comprises growing nanowires in the parallel grooves, wherein the nanowires have a diameter of less than about 10 nm.

In various embodiments, the forming comprises growing nanoribbons on the parallel steps, wherein the nanoribbons have a width of less than about 10 nm.

In various embodiments, the forming comprises shear-aligning randomly oriented nanowires or nanoribbons in a liquid suspension on the substrate by dragging an edge of a scraper through the liquid suspension.

In various embodiments, the nanowires or nanoribbons comprise two dimensional transition metal chalcogenide (TMD) semiconductor nanoribbons, carbon nanotubes, graphene nanoribbons, silicon nanoribbons, n-type doped silicon semiconductor nanoribbons, or p-type doped silicon semiconductor nanoribbons.

In various embodiments, the surface of the device structure comprises Si, SiO₂ on Si, or Al₂O₃ on Si.

In various embodiments, the first and second electrodes in each pair of electrodes comprise at least one of Au, Ag, Pd, Pt, Ru, Rh, Al, Cu, Ni, or alloys thereof.

In various embodiments, the openings in the dielectric layer are each sized to less than about 30 nm equivalent diameter.

In various embodiments, the openings are circular, each having a diameter of less than about 10 nm.

In various embodiments of the present disclosure, a method of manufacturing a sensor device comprises: forming a plurality of parallel aligned nanowires or nanoribbons on a substrate; transferring a group of the parallel aligned nanowires or nanoribbons from the substrate onto a surface of a device structure; disposing an array of pairs of electrodes on the surface in parallel such that each pair of electrodes electrically connects to one nanowire or nanoribbon, wherein each pair of electrodes in the array comprises a first electrode and a second electrode spaced apart from the first electrode by a nanogap, and wherein one nanowire or nanoribbon electrically connects the first and second electrodes in each pair of electrodes; patterning a dielectric layer over the parallel aligned nanowires or nanoribbons so as to leave one exposed region of nanowire or nanoribbon for each electrode pair, each exposed region positioned between the first and second electrodes in each pair of electrodes; and attaching a single molecule to each exposed region of nanowire or nanoribbon.

In various embodiments, the molecule comprises a DNA polymerase enzyme or an RNA polymerase enzyme.

In various embodiments, the forming comprises growing the nanowires or nanoribbons on parallel steps or within parallel grooves configured in the substrate.

In various embodiments, the forming comprises growing nanowires in the parallel grooves, wherein the nanowires have a diameter of less than about 10 nm.

In various embodiments, the forming comprises growing nanoribbons on the parallel steps, wherein the nanoribbons have a width of less than about 10 nm.

In various embodiments, the forming comprises shear-aligning randomly oriented nanowires or nanoribbons in a liquid suspension on the substrate by dragging an edge of a scraper through the liquid suspension.

In various embodiments, the nanowires or nanoribbons comprise two dimensional transition metal chalcogenide (TMD) semiconductor nanoribbons, carbon nanotubes, graphene nanoribbons, silicon nanoribbons, n-type doped silicon semiconductor nanoribbons, or p-type doped silicon semiconductor nanoribbons.

In various embodiments, the surface of the device structure comprises Si, SiO₂ on Si, or Al₂O₃ on Si.

In various embodiments, the electrodes in each pair of electrodes comprise at least one of Au, Ag, Pd, Pt, Ru, Rh, Al, Cu, Ni, or alloys thereof.

### BRIEF DESCRIPTION OF THE DRAWING FIGURES

The features and advantages of embodiments of the present disclosure will become more apparent from the detailed description set forth below when taken in conjunction with the drawings. The drawings and the associated descriptions are provided to illustrate embodiments of the disclosure and not to limit the scope of what is claimed.

FIGS. 1A and 1B (Design option #1) depict a side view for fabrication steps for MoS₂ ribbon bridge based biosensor, with size-limited, MoS₂ or TMD or semiconductor islands exposed, with optionally defective MoS₂, and any damage by nanopatterning repaired by annealing or plasma-treatment. (a) deposit and pattern conducting electrode pair (source and drain) and place a suspended MoS₂ layer, (b) To enable Nanoimprinting, planarize the surface, then use vertical Au nanopillar, and use size-limited MoS₂ region, (c) allow preferably a single molecule enzyme (DNA, RNA polymerase, etc.) to be attach on exposed MoS₂, (using biotin-streptavidin, antigen-antibody, peptide complex, etc.) for polymerase reaction of nucleotide attachment for FET sequencing, or other protein sensing.

FIGS. 2A and 2B (Design option #2) depict an exemplary design of single molecule bridge of DNA or RNA sensor comprising size-limited, MoS₂ type 2D chalcogenide semiconductor nano-ribbon region. The polymerase reaction of nucleotide attachment (nucleotide monomers like A,T,C,G, etc.) alters the electrical current properties of the FET molecular bridge on pulsing for sequencing analysis.

FIGS. 3A and 3B (Design option #3) depict an alternative design using temporary, removable guiding channel to allow a single polymerase attachment on MoS₂ nanoribbon bridge, but can be dissolved away later. (a) Removable tapered channel prepared by microfabrication or nanoimprinting, (b) Guided single polymerase attachment onto MoS₂ nanoribbon bridge, followed by dissolution removal of the sacrificial guide structure above.

FIGS. 4A and 4B schematically illustrate a cross-sectional view of producing diameter-reduced-tip Au electrode for size-confined molecular bridge sensor formation.

FIGS. 5A and 5B depict schematically a sectional view showing an example process of using a sacrificial plug to create a 5-10 nm, size-limited structure to place a single molecule polymerase sensor. Either nanoimprinting or sequential multilayer resists having gradually different dissolution rate to create a funnel shape type guidance geometry.

FIG. 6 provides an alternative method of guidance funnel shaping using nanoimprinting for 5-10 nm regime, in the PMMA or HSQ type resist to enable a placement of a single molecule polymerase sensor on a MoS₂ nanoribbon bridge. The protruding PMMA structure above also protects the attached polymerase from mechanically washed away by microfluidic solution flow or post-sequencing washing operation.

FIG. 7 illustrates use of additional force parameters to assist in guidance and placement of single polymerase or single biosensor molecule per each of the electrode array structure.

FIGS. 8A and 8B (Design option #4) depict an exemplary design of utilizing size-confined DNA assembly well to have only a “Single Streptavidin” immobilized for sequencing or protein sensing.

FIG. 9 (Design option #5) depicts a cross-sectional view of example design of utilizing shape memory type metal, ceramic or polymer functional material for temperature-, magnetic-, E-field-, pH- or chemical-responsive, dimension-changeable structure to physically trap only a “Single Streptavidin” or “single Polymerase” for high conductivity sequencing or protein sensing.

FIG. 10 depicts a top view of an array of TMD (like MoS₂ or WS₂) bridge molecular sensors, with a size-limiting structure to accommodate a single polymerase. Massively parallel electronic sequencing analysis can be performed with many devices organized into a system, having as many as 10,000 or even at least 1 million devices.

FIG. 11 depicts MoS₂ ribbon transfer deposited. A large sheet MoS₂ (e.g., >1 cm²) can be transfer deposited on CMOS chip or onto another device surface, and e-beam or nanoimprint patterned. For less time-consuming process and assembly, pre-slitted (but still frame-attached) MoS₂ could be transferred, or an array of nano-ribbons can be wet-stamp transferred onto the device surface, preferably using an elastomeric stamp (e.g., PDMS based), with optional binding-assisting thin layer of polymer type material, which can be later washed or burned away.

FIGS. 12A and 12B depict MoS₂ nano-ribbon array patterned on flat SiO₂ or other removable substrate surface, to be made transferable and releasable by a sequence of processes.

FIG. 13 depicts transfer of MoS₂ nanoribbons by PDMS type soft stamp. The PDMS stamp optionally has protruding ridges for easier pick up of the nanoribbons.

FIG. 14 depicts a top view of an array of Au electrode pairs (or other metal) with float-transferred or PDMS stamp-transferred MoS₂ bridges, with redundant ribbons to ensure at least one bridge formation occurs. Optionally, a masking coating with blocking agent may be added to prevent biotin, streptavidin or polymerase adhesion, except for a local ˜5 nm circle on MoS₂ surface, so as to ensure only a single enzyme molecule is attached.

FIG. 15 depicts: (a) Tethered array of encoded (memory written) DNA fragments periodically positioned on a substrate, (b) Polymerase-MoS₂ nanobridge array approaching DNA array being released, (c) DNA templates are taken up by polymerase array and the completed sensor array is moved array for DNA sequencing to read the recorded memory information. (Microfluidics chamber not shown). Massive parallel DNA written information array in combination with massively parallel MoS₂ nanobridge reader array allows ultrafast, “random-access-enabled” DNA memory retrieval. Several different configuration of tethered DNA memory array, with various methods of tethering and releasing, can be made to maximize the DNA memory processing capability.

FIG. 16 illustrates a sensor array fabrication method in accordance with various embodiments of the present disclosure, beginning with a silicon-on-insulator (SOI) wafer.

FIG. 17 illustrates various dimensions and shapes for semiconductor nanobridges in accordance with various embodiments, which are controllable, for example, by nanofabrication, nanoimprinting, shadow-mask RIE etch or by repeated oxidation/chemical etch.

FIG. 18 illustrates a method of alignment and attachment of floating nanoribbons comprising applying an electrical field across the electrode array to attract and attach one nanoribbon to each electrode pair.

FIG. 19 illustrates various embodiments of a method of preparing pre-aligned and parallel nanowires or nanoribbons utilizing a substrate having crystallographic steps.

FIG. 20 illustrates various embodiments of a method of preparing pre-aligned and parallel nanowires or nanoribbons utilizing a substrate having parallel configured grooves.

FIG. 21 illustrates various embodiments of a method of preparing pre-aligned and parallel nanowires or nanoribbons utilizing a scraper to drag through a liquid suspension of randomly oriented nanowires or nanoribbons.

FIG. 22 illustrates various embodiments of a method of utilizing pre-aligned and parallel nanowires or nanoribbons as a shadow mask.

FIG. 23 illustrates various methods of a stamp transfer process usable to move pre-aligned and parallel nanowires or nanoribbons onto a device structure.

FIG. 24 illustrates various methods of a stamp transfer process usable to move pre-aligned and parallel nanowires or nanoribbons onto a device structure already comprising an array of parallel aligned electrode pairs such that the nanowires or nanoribbons are transferred on top of the existing electrodes.

FIG. 25 illustrates in cross-section various embodiments of a complete sensor that is part of an array of sensors usable for DNA/RNA sequencing or for reading recorded memory information.

### DETAILED DESCRIPTION

Various aspects of the present disclosure generally provide sequencing apparatus, device structures, and methods for using two-dimensional, layer structured semiconductors usable to provide DNA and genome analysis performance. Various disclosed embodiments are amenable to scale-up processes in commercial manufacturing.

In various embodiments of the present disclosure, a biomolecular sensor comprises a nanobridge connected to spaced-apart electrodes and suspended over a gap between them. In various embodiments, the nanobridge comprises a transition metal dichalcogenide material or a silicon material. In various embodiments, a silicon material herein may include pure crystalline silicon or any type of doped silicon semiconductor material. Such materials may be obtained from silicon-on-insulator wafers.

Two dimensional (2D) layered materials such as transition metal dichalcogenides (TMDs) materials and devices have received much attention in recent years by virtue of their unique electronic, physical and chemical properties. One example is molybdenum dichalcogenide MoS₂ which can be incorporated as a sensor device. MoS₂ type 2D materials can be a single layered material or several layered material. The 2D layer materials such as MoS₂ can be produced by various known techniques, e.g., by isolation of very thin MoS₂ layer through mechanical exfoliation, physical or chemical vapor deposition, molecular beam epitaxy (MBE) type construction, or sulfurization of a transition metal layer such as Mo or W.

### Definitions

As used herein, the term “nucleotide” means either the native dNTPs like A, T, C, G (i.e., dATP, dTTP, dCTP and dGTP), or collectively refers to various types of modified dNTPs.

As used herein, the term “polymerase” means an enzyme that synthesizes long chains or polymers of nucleic acids. For example, DNA polymerase and RNA polymerase can copy a DNA or RNA template strand, respectively, using base-pairing interactions, thus assembling DNA and RNA molecules.

TMD Layers and Combined TMD Materials for Sensor Bridges

In various embodiments, a TMD layer is incorporated as a part of sensor bridge structure to attach an enzyme type biomolecule to attract various types of nucleotides for electronic detection signals.

Two dimensional transition metal dichalcogenide (TMD) monolayers are in general atomically thin semiconductors of the type MX₂, with M a transition metal atom (notably including Mo, W, or Ti, Zr, Hf, V, Nb, Ta, Tc, Re, Co, Rh, Ir, Ni, Pd, Pt) and X a chalcogen atom (such as S, Se, or Te). One layer of M atoms is sandwiched between two layers of X atoms. Both the transition metal and the chalcogenide element can be partly replaced (or doped) with other elements. Therefore, the two dimensional TMD layer incorporated into the molecular sensor bridge construction can have various modified or altered composition ranges, including the following:

(i) MoS₂, WS₂, or TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations, including modified stoichiometry of sulfur contents having MX₍₂₋ₓ₎ or MX₍₂₊ₓ₎ with the desired value of x in the range of 0-1.0, preferably in the range of 0-0.5, even more preferably 0-0.3. For various embodiments, the sulfur stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(ii) MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations, including modified stoichiometry of selenium contents having MX₍₂₋ₓ₎ or MX₍₂₊ₓ₎ with the desired value of x in the range of 0-1.0, preferably in the range of 0-0.5, even more preferably 0-0.3. For various embodiments, the selenium stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(iii) MoTe₂, WTe₂, or TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂ and their modifications or combinations, including modified stoichiometry of tellurium contents having MX₍₂₋ₓ₎ or MX₍₂₊ₓ₎ with the desired value of x in the range of 0-1.0, preferably in the range of 0-0.5, even more preferably 0-0.3. For various embodiments, the tellurium stoichiometry is intentionally altered so as to provide vacancy defects, interstitial defects, and aggregated defects so as to increase the surface energy and enhance the adhesion of biomolecule to the bridge sensor. Such defects can also increase the bandgap for stronger sensor signals;

(iv) Mixed TMD compounds in which the MX₂ compound has mixed metals and/or mixed chalcogenide. For example Mo(SₓSe_{y}Te_{z})₂, W(SₓSe_{y}Te_{z})₂, or Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂ where the combined (x+y+z) is 1-3, preferably 0.5-1.5, even more preferably 0.7-1.3. Alternatively, two or more metals can be combined for sulfur containing, Se-containing or Te-containing TMD layers, e.g., (MoₓW_{y}Co_{z})S₂, (HfₓW_{y}Co_{z})Te₂ and so forth; or

(v) M_{(1-w)}N_{y}X_{(2-z)}Y_{z} structure in which the transition metal M is partially substituted with non-transition elements N, with a concentration of w and the N element selected from one or more of Al, Si, Ga, Ge, In, Sn, Sb, Bi, Al, Na, K, Ca, Mg, Sr, Ba, with the w value in the range of 0-0.3, and the chalcogenide element X partially substituted with a non-chalcogenide element Y, with the Y element selected from one or more of Li, B, C, N, O, P, F, Cl, I, with the z value in the range of 0-0.3.

In various embodiments, the thickness of a MoS₂ monolayer can be about 6.5 Å. The TMD materials in their simplest monolayer structure, e.g., MoS₂, WS₂, MoSe₂, WSe₂, MoTe₂, have a direct band gap, and hence can be used in electronics as transistors or sensors. Either monolayer TMD or few layer TMD can be structurally modified, to be utilized as solid state DNA or genome sensors, without labeling with optical capability. Being an ultrathin direct bandgap semiconductor, a transition metal dichalcogenide such as single-layer MoS₂ has found some useful applications in nanoelectronics, optoelectronics, and energy harvesting. However, not many sensor applications have been attempted or demonstrated with proper characteristics, especially for DNA or genome sequencing purposes.

Silicon Layers for Sensor Bridges

In various embodiments, a biosensor herein may comprise a nanobridge made of a silicon material rather than a TMD material. As used herein, “Si-material” or “Si-type” bridge, nanobridge or layer may comprise pure crystalline silicon or any type of doped silicon semiconductor material, as described herein.

In various embodiments, a silicon nanobridge may comprise a semiconductor wire or a ribbon structure. In various embodiments, a silicon nanobridge may comprise material derived from a silicon-on-insulator (SOI) wafer, and may take the form of a nanostructure or related semiconductor nanoribbon or nanowire structure.

A single nanobridge structure of crystalline silicon may be obtained from silicon-on-insulator (SOI) semiconductor substrate (or other related semiconductor layers), which can be made into a reliable nanobridge sensor structure since the material is already properly-doped semiconductor films with such a nanobridge material being less sensitive to nanopatterning-related damages. Therefore, a need for high temperature processing is minimized. Unique SOI-based fabrication of nanobridge structures, processing methods and applications of such a single-bridge SOI-based nanoribbon biosensors are disclosed in the drawing figures.

The thickness of a doped SOI Si thin film nanobridge is desirably thin, e.g., <30 nm, preferably <20 nm thick, more preferably <10 nm thick layer, and even more preferably <5 nm, with a width of <40 nm, preferably <20 nm, more preferably <10 nm, and even more preferably <3 nm. A smaller cross-section nanowire or nanoribbon bridge across two mating electrodes can provide higher signal to noise ratio during sequencing interactions.

Also, a smaller cross-section nanowire or nanoribbon bridge of SOI-based Si nanobridge or crystalline semiconductors in general (e.g., <10 nm width, preferably <5 nm width, more preferably 3 nm width), ensures that only a single (or at most a few) nanobridge structures will attach to form one or at most a few bridges, due to size exclusion, instead of multiple bridges, with the latter introducing multiple, undesirably complicated signals from the parallel bridges stuck together on the same electrode lead pair. A single and narrower sensor bridge will also prefer a single-molecule polymerase placement for each electrode pair.

Drastically narrowed SOI-Si structure for a single nanobridge can be prepared using highly advanced nanofabrication of Si and SiO₂ structures. Another approach to further ensure a single bridge formation of SOI-Si, according to various aspects of the present disclosure, is to utilize a nano-mask array, for example, by placing an array of e.g., <5 nm wide graphene, carbon, ceramic or metallic nanoribbon mask so that the SOI-Si underneath can be patterned and shaped to become narrow nanobridges. Such pre-made narrowed graphene, carbon, ceramic or metallic nanoribbon mask can be prepared on another substrate followed by stamp transfer using a PMMA (Polymethyl methacrylate), PDMS (Polydimethylsiloxane) or other polymeric or elastomeric stamp materials. Such pre-made narrowed bridge can be used as a sensor bridge on which a single enzyme molecule such as polymerase can be attached.

In various embodiments, such SOI-Si type or other semiconductor ribbon strips can be split into two nanobridges between which another molecular bridge (such as a single DNA or peptide) can be attached for ease of polymerase bonding and nucleotide analysis. Such splitting of amorphous semiconductor into two separated parts with a nanogap in between (e.g., 20-100 nm) can be accomplished by e.g., focused laser beam slicing, focused ion beam cutting or patterning and etching. The ends of the split ribbons facing each other can desirably be sharpened to a pointed-tip geometry of e.g., 2-5 nm radius of curvature, so as to facilitate an attachment of a single DNA or a single peptide molecular bridge, using either electric field alignment, flow alignment in a microfluidic chamber, or stamp transfer of pre-aligned DNA or peptide. Stamp transfer of pre-aligned DNA or peptide array can be made using PMMA, PDMS or other polymer type soft stamps.

Biosensors

Disclosed herein are label-free DNA or RNA sequencing device structures utilizing a TMD- or Si-based frame with an enzyme polymerase for detection of electronic signals when an individual nucleotide is attached onto a nucleic acid template. In various embodiments, two dimensional semiconductors of processed, defective or nanoporous Transition Metal Dichalcogenide (TMD) layer material are employed so as to utilize altered bandgaps of the TMD layer and enhanced attachment of single biomolecules. In various embodiments, the TMD-based sequencing systems disclosed herein can be assembled into a massively parallel configuration for rapid analysis of targets including nucleotides, in particular for applications of sequencing of a DNA molecule, or a collection of such molecules constituting an entire genome. Such systems are also useful for DNA-based information storage, for which the writing is performed by encoding specific nucleotide-based arrangements or sequences and the reading is carried out by sequencing analysis using TMD-bridge based molecular sensor array.

A bridge-configured sensor structure comprising an elongated nano-dimension, crystalline semiconductor wire or ribbon, such as silicon or doped silicon, is another way of producing label-free molecular sensors for genome sequencing without introducing complicated fluorescence imaging methodologies. Such semiconductor nanowires of, e.g., made of Si derived from silicon-on-insulator (SOI) wafer, can be connected to a pair of electrodes (with optional gate structure) in high density electronic circuit assembly, also equipped with microfluidic environment comprising floating DNAs, nucleotides, enzyme polymerase molecules, etc. It is possible to attach a polymerase single molecule (or few molecules) to such a nanobridge or elongated biomolecule bridge, using, for example, functionalities and ligands such as biotin-streptavidin, antibody-antigen, pyrene or peptide complexes.

Inorganic nanobridges, either van der Waals force connected or metallization connected to the device electrode leads can offer much higher electrical conductivity and substantially higher electrical sensor signals than organic nanobridges as the use of undesirably high electrical resistance ligands or attaching functionalities can be minimized.

In various embodiments of the present disclosure, biosensor bridges connected between a pair of conducting electrodes (such as made of Au, Ag, Pd, Pt, Ru, Rh, Al, Cu, Ni, etc.), can be accomplished by an active attachment route using electrical field (electrophoretic alignment and attachment using various AC or DC mode of electric fields, often at a level of 0.2 to 10 volts). Alternatively, aligned nanobridge structures connecting the mating electrodes can also be achieved by stamp transfer of pre-aligned (or pre-patterned) inorganic nanobridge array from another substrate using PMMA, PDMS or other stamp materials. On the device substrate or temporary preparation substrate, the nanobridge array in parallelly aligned configuration is prepared either by nanopatterning by lithographic means, flow alignment in a microfluidic chamber, or by electrical field alignment (e.g., dielectrophoretic alignment using AC or DC electric field).

While a number of nanomaterials can be considered for such nanobridge structures, these nanomaterials need to be processed into nano-dimension wires or ribbons by nanolithography or other treatments, which tends to cause damage to crystallographic structures or disruption of atomic arrangements causing unintended changes or deterioration of physical or electronic properties. While a high temperature annealing process (e.g., 500° to 1000° C.) can sometimes repair or reduce such damages, the use of high temperature processing steps often damages electronic device structure and hence should be avoided for ease of device manufacturing and reliability.

For accurate signal detection on nucleotide attachment events (or other biomolecule attachment events, e.g., to polymerase, to enable electrical signal detection for genome sequencing), it is highly desirable to provide a single elongated bridge (made of inorganic or organic nanowires or nanoribbons) between mating electrodes (made of Au, Pd or other conducting lead wires). If multiple nanobridges are attached between the two mating electrodes, often clumped together, multiple signals may occur by the presence of parallel sensors, which makes the analysis of such complicated signals very difficult.

Information Storage

DNA data storage is a process of digital encoding and decoding binary data, to and from synthesized or duplicated DNA strands. For example, the binary code information storage of (00), (01), (10) and (11) can be replaced by various arrangements of oligonucleotides (A, C, G, T). DNA molecules are genetic blueprints for living organisms, and the information stored in DNA is known to last more than 10,000 years under certain environment. With its huge capacity (many orders of magnitude larger than what is possible with current technology) to store enormous amount of information in very small space, DNA storage could be the answer to a modern era problem of too much information that needs to stored, e.g., on the order of hundreds of zettabytes every year in the near future. Currently available information storage capability including magnetic disk, tape, optical or other related technologies can cover only a fraction of such a needed capacity.

While substantial progress has been made in DNA information storage in recent years, cost effective data storage techniques for practical applications are yet to be achieved. For efficient retrieval of stored information, the encoded DNA nucleotide arrangements need to be decoded by, e.g., reading (or sequence analysis). A fast, economic method of reading the encoded DNA information is essential for the success of DNA based data storage. This disclosure also provides new methods and device structure to enable such progress.

Biosensors having a TMD or silicon nanobridge provide reliable DNA genome analysis performance and are more easily amenable to scalable manufacturing, because the need for high temperature processing is minimized. The disclosed structures herein are also useful for DNA-based large-capacity information storage devices including archival or randomly-accessible-memory and logic devices.

TMD Compositions

In various embodiments, a sequencing device is provided comprising: (a) an electrode array of conducting electrode pairs, each pair of electrodes comprising a source and a drain electrode separated by a nanogap, said electrode array deposited and patterned on a dielectric substrate, and optionally comprising a third electrode as a gated system; (b) a single-layer or few-layer thick transition metal dichalcogenide (TMD) layer disposed on each pair of electrodes, connecting each source and drain electrode within each pair, and bridging each nanogap of each pair; (c) a dielectric masking layer disposed on the TMD layer and comprising size-limited openings that define exposed TMD regions, each opening sized so as to allow only a single enzyme biomolecule to fit therein and to attach onto the exposed TMD region defined by each opening; (d) an enzyme molecule attached to each exposed TMD region such that only one enzyme molecule is found within each opening; and (d) a microfluidic system encasing the electrode array, wherein attachment or detachment of a biomolecule selected from the group consisting of a nucleotide monomer, a protein, and a DNA segment, onto the enzyme molecule, one at a time, can be monitored as a uniquely identifiable electrical signal pulse to determine the specific nature of the biomolecule attaching or detaching.

In various embodiments, the dielectric substrate comprises SiO₂. In various embodiments, the dielectric substrate comprises SiO₂ or Al₂O₃.

In various embodiments, the TMD is selected from MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations. In various embodiments, the TMD is MoS₂. In various embodiments, the TMD is WS₂.

In various embodiments, the TMD is selected from MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations, including modified stoichiometry of sulfur contents having MX₍₂₋ₓ₎ or MX₍₂₊ₓ₎ wherein x is in the range of 0-1.0, preferably in the range of 0-0.5, even more preferably 0-0.3. In various embodiments, the TMD is selected from MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂ and their modifications or combinations and the stoichiometry of sulfur is not modified.

In various embodiments, the TMD is selected from MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations, including modified stoichiometry of selenium contents having MX₍₂₋ₓ₎ or MX₍₂₊ₓ₎ with the desired value of x in the range of 0-1.0, preferably in the range of 0-0.5, even more preferably 0-0.3. In various embodiments, the TMD is selected from MoSe₂, WSe₂, or TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂ and their modifications or combinations and the stoichiometry of selenium is not modified.

In various embodiments, defects are artificially introduced into TMD. In various embodiments, the defects are introduced to increase bandgap. In various embodiments, the defects are introduced to provide active site edge locations for strong adhesion of bridge structures or biomolecules such as enzyme molecules.

In various embodiments, TMD is selected from MoTe₂, WTe₂, or TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂ and their modifications or combinations.

In various embodiments, TMD is selected from MoTe₂, WTe₂, or TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂ and their modifications or combinations, including modified stoichiometry of Tellurium contents having MX₍₂₋ₓ₎ or MX₍₂₊ₓ₎ with the desired value of x in the range of 0-1.0, preferably in the range of 0-0.5, even more preferably 0-0.3. In various embodiments, TMD is selected from MoTe₂, WTe₂, or TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂ and their modifications or combinations and the stoichiometry of tellurium is not modified.

In various embodiments, the tellurium stoichiometry is intentionally altered so as to provide vacancy defects, interstitial defects, and aggregated defects in order to increase the surface energy of the TMD layer and enhance the adhesion of biomolecule to the bridge sensor for stronger sensor signals.

In various embodiments, the TMD comprises a mixed TMD selected from TMD compounds in which the MX2 compound has mixed metals and/or mixed chalcogenide, selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, and Pt(SₓSe_{y}Te_{z})₂ wherein the combined (x+y+z) is 1-3, 0.5-1.5, or 0.7-1.3.

In various embodiments, two or more metals are combined for sulfur containing, Se-containing or Te-containing TMD layers.

In various embodiments, the TMD layer comprises (MoₓW_{y}Co_{z})S₂ or (HfₓW_{y}Co_{z})Te₂.

In various embodiments, the TMD comprises a M_{(1-w)}N_{y}X_{(2-z)}Y_{z} structure in which the transition metal M is partially substituted with non-transition elements N, with a concentration of w and the N element selected from one or more of Al, Si, Ga, Ge, In, Sn, Sb, Bi, Al, Na, K, Ca, Mg, Sr, Ba, with the w value in the range of 0-0.3, and the chalcogenide element X partially substituted with a non-chalcogenide element Y, with the Y element selected from one or more of Li, B, C, N, O, P, F, Cl, I, with the z value in the range of 0-0.3. In various embodiments, the w value is greater than 0.3, for example, greater than 0.4, greater than 0.5, greater than 0.6, greater than 0.7, greater than 0.8, greater than 0.9, or greater than 1.0. In various embodiments, the z value is greater than 0.3, for example, greater than 0.4, greater than 0.5, greater than 0.6, greater than 0.7, greater than 0.8, greater than 0.9, or greater than 1.0.

Silicon Compositions

In various embodiments, a sequencing device is provided comprising: (a) an electrode array of conducting electrode pairs, each pair of electrodes comprising a source and a drain electrode separated by a nanogap, said electrode array deposited and patterned on a dielectric substrate, and optionally comprising a third electrode as a gated system; (b) a single-layer or few-layer thick silicon or doped silicon semiconductor layer disposed on each pair of electrodes, connecting each source and drain electrode within each pair, and bridging each nanogap of each pair; (c) a dielectric masking layer disposed on the silicon layer and comprising size-limited openings that define exposed silicon regions, each opening sized so as to allow only a single enzyme biomolecule to fit therein and to attach onto the exposed silicon region defined by each opening; (d) an enzyme molecule attached to each exposed silicon region such that only one enzyme molecule is found within each opening; and (d) a microfluidic system encasing the electrode array, wherein attachment or detachment of a biomolecule selected from the group consisting of a nucleotide monomer, a protein, and a DNA segment, onto the enzyme molecule, one at a time, can be monitored as a uniquely identifiable electrical signal pulse to determine the specific nature of the biomolecule attaching or detaching.

In various embodiments, the electrodes in each electrode pair may comprise Au, Ag, Pd, Pt, Ru, Rh, Al, Cu, Ni, etc.

In various embodiments, the dielectric substrate comprises SiO₂. In various embodiments, the dielectric substrate comprises SiO₂ or Al₂O₃.

In various embodiments, the silicon material forming the nanobridge comprises pure crystalline silicon, such as single crystalline silicon.

In various embodiments, the silicon material forming the nanobridge comprises an n- or p-doped silicon semiconductor material. Dopants for n-type or p-type doped silicon semiconductor may comprise acceptors from Group III elements or donors from Group V elements. Dopants for n- or p-doped silicon semiconductor include, but are not limited to As, B, P, Sb, Ga, Zn, and Fe.

In various embodiments, a silicon nanobridge may be obtained from a SOI wafer manufactured with the desired silicon material (e.g., single crystalline silicon, n-type silicon semiconductor, or p-type silicon semiconductor).

Various aspects of the present disclosure provide biosensor structures, materials and geometries, as well as fabrication methods and application methods, such as described below in reference to the various drawing figures.

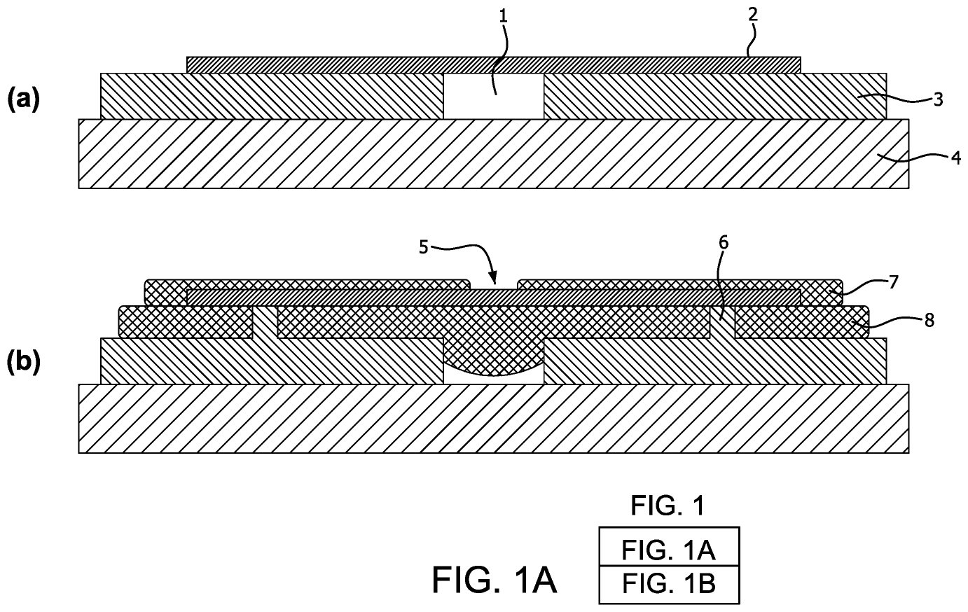

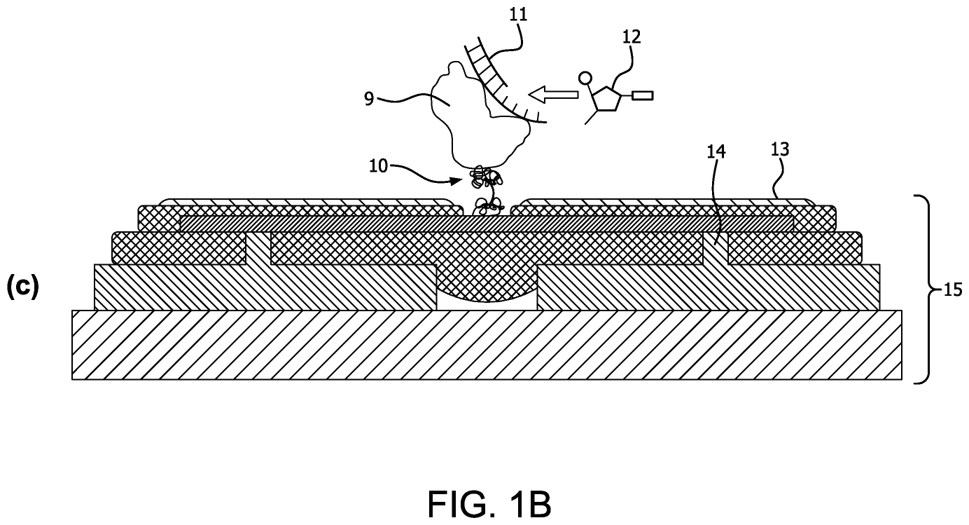

FIGS. 1A-1B illustrate various sensor structures and manufacturing steps for making a complete sensor in accordance with various aspects of the present disclosure. In general, structure (a) shows a nanoribbon over a nanogap; structure (b) shows a nanoribbon on a planarize surface; and structure (c) shows a size limited nanoribbon wherein a dielectric coating obscures most of the nanoribbon, leaving only a small exposed region of nanoribbon that fits only a single biomolecule, like an enzyme molecule.

FIG. 1A(a) shows a conducting electrode pair 3 disposed on a dielectric substrate 4. There may be a plurality of pairs of electrodes, such as an array; however for clarity only the cross-section of one pair of electrodes is shown. The electrodes 3 may comprise conducting metals or alloys, such as Au, Ag, Pd, Pt, Ru, Rh, Al, Cu, Ni, etc., or their various alloys. The spacing 1 between electrodes is referred to as the nanogap 1, which may be dimensioned from about 2 nm to 100 nm, or 2 nm to about 20 nm, and preferably from about 5 nm to about 20 nm. The underlying substrate 4 may comprise such materials as SiO₂, or SiO₂ on Si. FIG. 1A(a) also shows a suspended TMD (e.g., MoS₂) or Si-material nanoribbon 2 (or other semiconductor nanobridge), having a rectangular, or narrow-ribbon configuration. Such a layer may be nanopatterned by nanoimprinting or e-beam lithography, or applied onto the electrodes and over each nanogap by a stamp transfer process, as explained below. The TMD material may comprise any transition metal dichalcogenide as disclosed herein. The Si-material for the Si nanoribbon may comprise pure single crystal silicon or any p- or n-type doped silicon semiconductor, such as obtained from an SOI wafer.

FIG. 1A(b) shows a device structure comprising conducting pillars 6, that in various aspects function as vertical extensions of the underlying electrodes. The pillars may comprise the same material as the electrodes, such as the same metal, or the pillars may comprise deposits of a different material. As shown, the device structure (b) comprises a size-limited (e.g., 5 nm dia.), exposed MoS₂ or other TMID or Si-material region 5, appropriately sized to accommodate only a single biomolecule (e.g., a polymerase enzyme) for binding to the exposed portion of the nanobridge. That is, the opening 5 is dimensioned so as to allow only a single biomolecule, such as a single polymerase enzyme, to contact and attach to the exposed TMD or Si-material bridge. The coating of the majority of the TMD or Si-material bridge, leaving only the opening 5, may be accomplished by a size-limiting dielectric coating 7 (PMMA or SiO₂) disposed over the TMD or Si-material bridge 2. Alternatively, a pre-planarized PMMA layer 8 provides a flat surface for nanoimprinting or other uses. The nanopillars 6 may comprise vertical Au nanopillars (about 5 nm-50 nm diameter), or may comprise other metals, such as Pd, Pt, Ru, Rh, etc. In various embodiments, the pillars may comprise island structures rather than cylinders, e.g., island Au pads, like hemispheres or other structures having at least a pillar top surface region.

In various embodiments, the metallic conducting electrode pair is selected from Au, Ag, Pd, Pt, Ru, Rh, Al, Cu, Ni, or their alloys. In various embodiments, the conducting electrode pair 3 may comprise Al, Cu, Ru, Pt or Pd, whereas the pillars 6 may comprise Ru, Pt, or Pd.

In various embodiments, the nanogap 1 is from about 5 nm to about 20 nm. In various embodiments, the nanogap is less than 5 nm, for example less than 3 nm, or less than 1.0 nm. In various embodiments, the nanogap is greater than 20 nm, for example, greater than 25 nm, greater than 30 nm, greater than 35 nm, greater than 40 nm, greater than 45 nm, or greater than 50 nm.

In various embodiments, the size-limiting openings 5 are preferably less than 30 nm average equivalent diameter each, more preferably less than 20 nm equivalent diameter, even more preferably less than 10 nm equivalent diameter. The openings 5 can be fashioned by lithographic or nanofabrication defined coverage of a dielectric material layer, polymer or ceramic in all the areas outside a specific, size-limited region intended for attaching only a single molecule. The term “average equivalent diameter” is used when the openings 5 are not circular, but where the calculated surface area can be expressed as though the openings are circular. Polymerase molecules, as well as streptavidin-type linked molecules, often have a steric size on the order of ˜5 nm regime. With the openings 5 thus configured, only one biomolecule fits within each opening to attach to the exposed TMD or Si-material inside the opening.

Pre-Aligned Nanoribbons and Nanowires for Sensor Use

As discussed above, substantially narrowed nanowires or nanoribbon structures can be attached to electrode pairs to form arrays of structures wherein each structure comprises a pair of spaced apart electrodes connected by a single nanowire or nanoribbon that resides along the substrate on which the electrodes are disposed, or that resides across the electrodes to form a bridge over the nanogap between the two electrodes in each pair of electrodes.

While current molecular electronic devices can electronically measure molecules for various applications, they lack the reproducibility as well as scalability and manufacturability needed for rapidly sensing many analytes at a scale of up to millions in a practical manner. Such highly scalable methods are particularly important for DNA sequencing applications, which often need to analyze millions to billions of independent DNA molecules. In addition, the manufacture of current molecular electronic devices is generally costly due to the high level of precision needed.

In various embodiments, new and improved sequencing apparatus and associated sensor configurations and methods comprise advanced elongated bridge structures. These bridge structures may comprise silicon-on-insulator (SOI) nanostructures or related semiconductor nanoribbons or nanowires, providing reliable DNA genome analysis performance. The structures are amenable to large scale manufacturing as the need for high temperature processing is minimized. The disclosed structures are also useful for DNA-based large-capacity information storage devices including archival or randomly accessible memory and logic devices.

To manufacture complete arrays of these structures, nanowires or nanoribbons may be formed in parallel arrays such that a plurality of nanowires or nanoribbons are pre-aligned, or a plurality of nanowires or nanoribbons may be aligned in a parallel configuration after formation. Following formation of a plurality of parallel aligned nanowires or nanoribbons, a group of the parallel aligned nanowires or nanoribbons is picked up and transferred onto device substrates that already have the pairs of electrodes, or alternatively, is transferred onto device structures after which electrode pairs are deposited. These two routes provide devices comprising nanowires or nanoribbons bridging the electrode pairs and devices having the nanowires or nanoribbons directly on the device substrate and not bridging the electrode pairs, respectively. Parallel oriented nanowires or nanoribbons may be transferred in groups comprising >2, or >5, or >10, or >100, or >1,000, or >10,000, or >100,000, or >a million nanowires or nanoribbons, while maintaining the parallel orientation of the group.

In various embodiments, a bridge-configured sensor structure comprising an elongated nano-dimensioned semiconductor wire or ribbon is one way of producing label-free molecular sensors for genome sequencing without introducing complicated fluorescence imaging methodologies. Such semiconductor nanowires may comprise organic nanowires such as DNA, peptide or their assemblies, or inorganic nanowires or nanoribbons e.g., carbon nanotubes (CNTs), single-walled CNTs (SWCNTs), graphene or MoS₂, Si- or doped silicon, or other semiconductor materials. Such nanowires or nanoribbons can be connected to a pair of electrodes (with optional gate structure) in various high density electronic circuit assemblies surrounded by a microfluidic environment comprising floating DNAs, nucleotides, enzyme polymerase molecules, and so forth to self-assemble various elements of a working sensor device and to operate it. For example, a single polymerase enzyme molecule (or just a few molecules) may be attached to such a nanobridge or other elongated biomolecule, e.g., by using functionalities and ligands like biotin-streptavidin, antibody-antigen, pyrene or peptide complexes, or other binding linkages between enzyme and nanowire or nanoribbon.

In various embodiments, inorganic nanobridges, either van der Waals force connected or metallization connected to the device electrodes, offer much higher electrical conductivity and substantially higher electrical sensor signals than organic nanobridges as the use of undesirably high electrical resistance ligands or linking functionalities can be minimized.

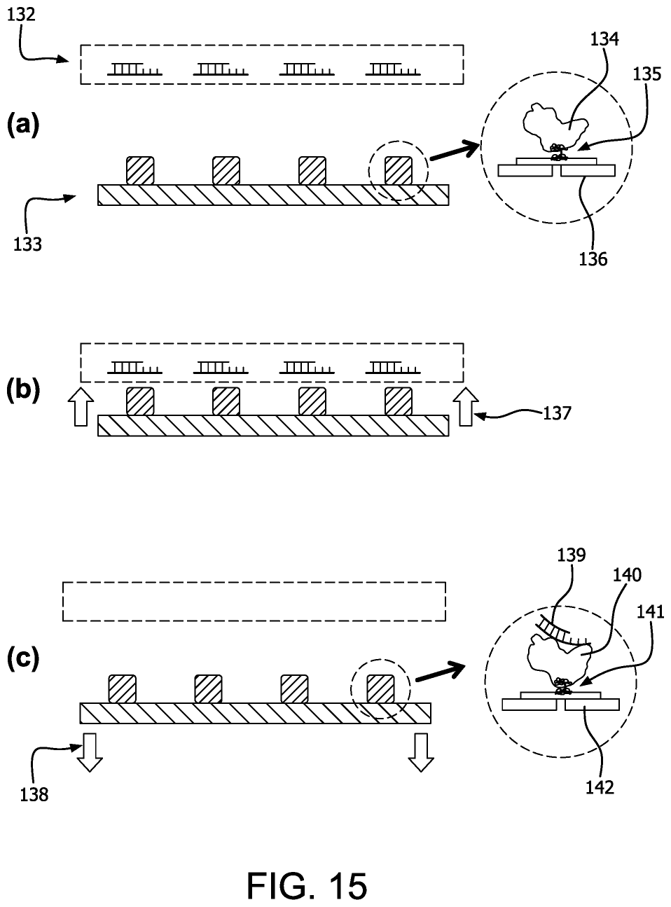

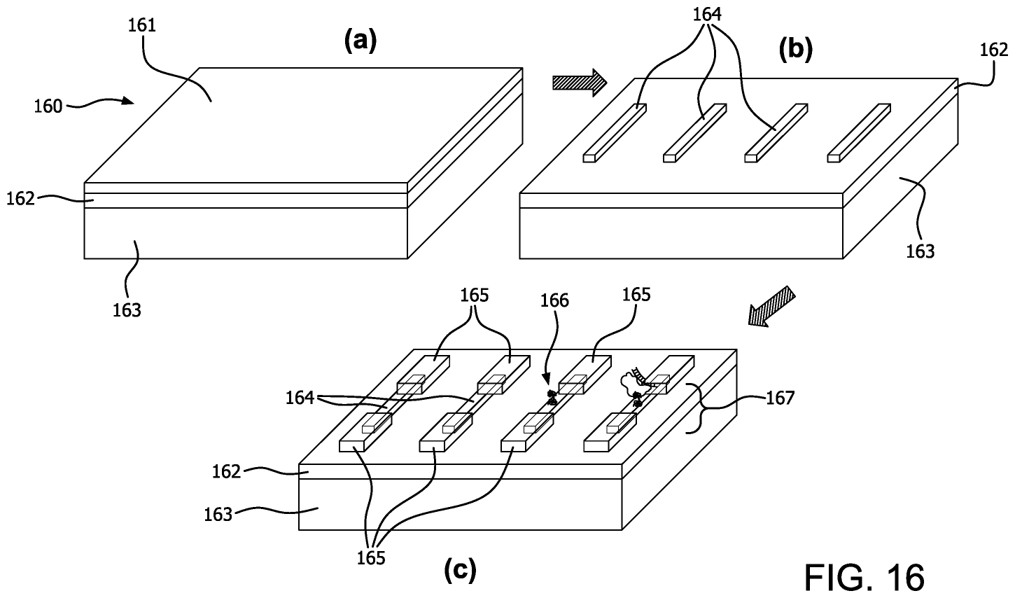

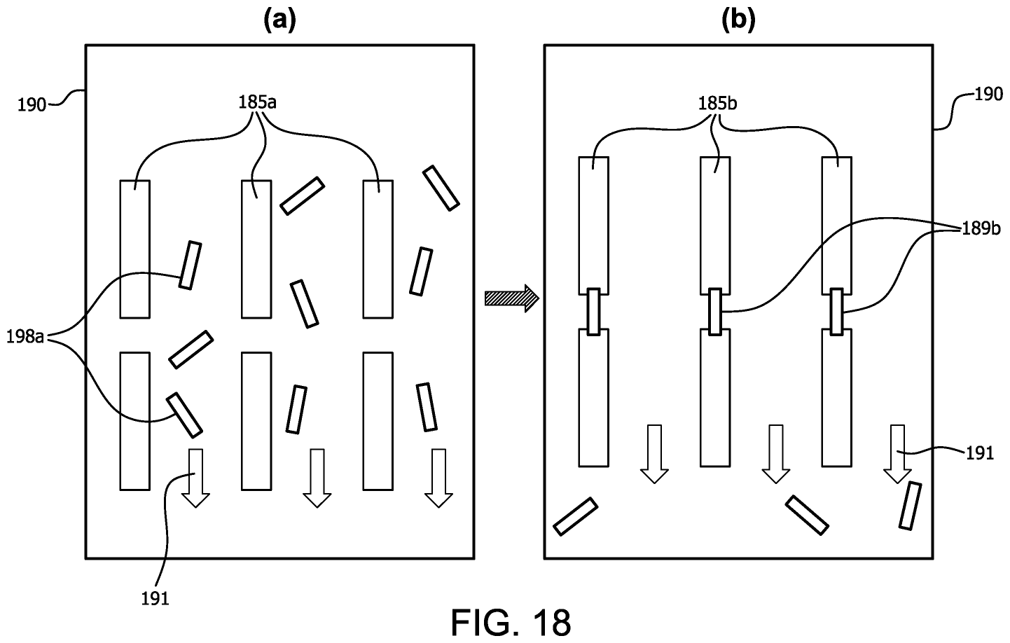

In various embodiments of the present disclosure, a biosensor bridge between a pair of conducting electrodes (e.g., Au, Ag, Pd, Pt, Ru, Rh, Al, Cu, Ni, or their alloys, etc.), can be accomplished by an active attachment route using electrical field (electrophoretic alignment and attachment using various AC or DC mode of electric fields, often at a level of 0.2 to 10 volts, as per FIG. 18). Alternatively, aligned nanobridge structures connecting the mating electrodes can also be achieved by stamp transfer of pre-aligned (or pre-patterned) inorganic nanobridge array from another substrate using PMMA, PDMS adhesive polymer, or other stamp materials (e.g., per FIGS. 15, 23 and 24). On the device substrate or temporary preparation substrate, the nanobridge array in parallelly aligned configuration is prepared either by nanopatterning by lithographic means, flow alignment in a microfluidic chamber, various shear alignment techniques, or by electrical field alignment (e.g., dielectrophoretic alignment using AC or DC electric field).

In various embodiments, a number of nanomaterials can be considered for such nanobridge structures. However, these nanomaterials need to be processed into nano-dimension wires or ribbons by nanolithography or other treatments that can cause damage to crystallographic structures or disruption of atomic arrangements causing unintended changes or deterioration of physical or electronic properties. An optional high temperature annealing process (e.g., at 500 to 1000° C.) can often produce well annealed nanowires or nanoribbons with more desirable properties. Such high temperature processes also tend to repair or reduce defects or damages in the nanowire or nanoribbon structure.

In various embodiments, accurate signal detection corresponding to nucleotide-polymerase enzyme attachment events (or other biomolecule attachment events), e.g., electrical signal detection for genome sequencing, benefits from having a single elongated bridge (inorganic or organic nanowires or nanoribbons) between mating electrodes. If multiple nanobridges are attached between the two mating electrodes in a pair of electrodes, often clumped together, multiple electrical signals may occur by the presence of parallel sensors, making analysis of the signals very difficult.

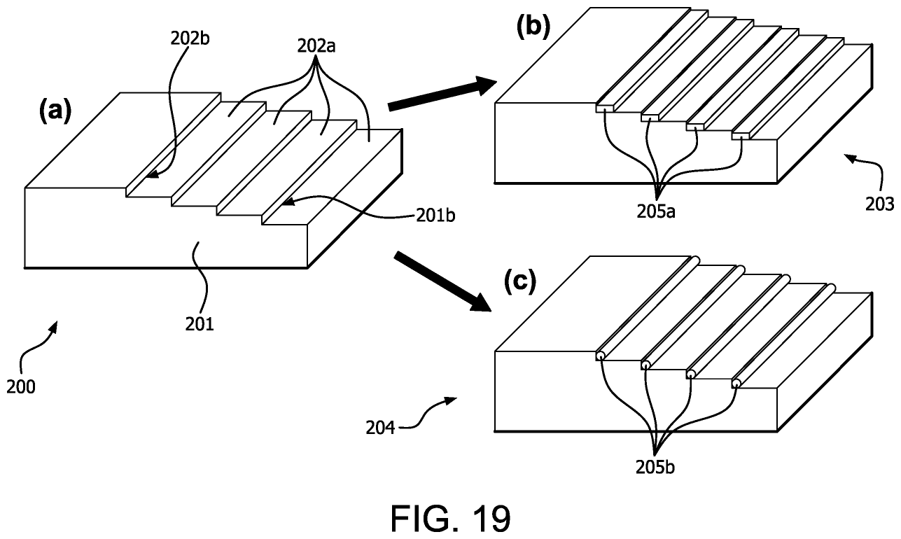

In various embodiments, innovative approaches are disclosed for producing a single nanobridge structure of nanoribbon array of Si or other crystalline semiconductors, band-gap opened graphene or MoS₂ or WS₂ type two-dimensional chalcogenide semiconductors, or a nanowire array such as semiconducting carbon nanotubes, ZnO, InP, or GaAs based nanowires. Unique pre-aligned fabrication of nanoribbons or nanowires on parallel configured step-edge sites or pre-established nano-groove sites on a growth substrate such as a cut crystal, including deposition substrate or alignment substrate, enables a transfer of such aligned nanowires or nanoribbons from the growth substrate onto a device electrode surface. The transferred nanowires or nanoribbons can optionally anchor as robust bridge structures, using dielectric or metallic coatings on regions away from the nanobridge location. Processing methods, structures and applications of such a single-bridge nanoribbon or nanowire biosensors are also disclosed as described in the drawings (e.g., FIGS. 19-24) as discussed below.

In various embodiments, aligned nanowires or nanoribbons according to the present disclosure can also be utilized as a mask to define and fabricate nanoribbons of a thin sheet underneath, e.g., graphene or 2D chalcogenide films such as MoS₂ or WS₂. Reactive ion etch (RIE etch) or oxygen plasma etch methods can be employed to synthesize such nanoribbons based on a nano-shadow-mask approach.

In various embodiments, the thickness of a nanoribbon or nanowire for use in a single-molecule sensor is about <30 nm, preferably <20 nm, more preferably <10 nm, and even more preferably <5 nm). The width of a nanoribbon or nanowire for use in a single-molecule sensor is about <40 nm, preferably <20 nm, more preferably <10 nm, and even more preferably <5 nm. A smaller cross-section nanowire or nanoribbon bridge across two mating electrodes in a pair of electrodes can provide higher signal-to-noise ratio during sequencing interactions such as nucleotide/polymerase interaction. Herein, these small cross-sectional nanoribbons or nanowires are referred to as being “substantially narrowed.” In various embodiments, a substantially narrowed nanowire has a diameter of about 5 nm and thus a cross-sectional area of only about 20 nm².

In various embodiments, substantially narrowed nanoribbons and nanowires are prepared using site-controlled synthesis of elongated and aligned geometry that can be transferred to form a FET device.

In various embodiments, nanoribbons or nanowires are shear-aligned. For this method, nanoribbons or nanowires may be randomly oriented in a liquid and then aligned by the action of a scraper having a flat, sawtooth, or other grooved edge configuration.

In various embodiments, single nanobridge structures are produced from a nanoribbon array obtained from floating semiconductors in a liquid medium. Examples of semiconductors that can be aligned or fabricated using the methods in this invention include, but are not limited to, Si, doped silicon, ZnO, GaAs, InP or other crystalline semiconductor nanowires or nanoribbons, amorphous semiconductor nanowires or nanoribbons, band-gap opened graphene or MoS₂ or WS₂ type two-dimensional chalcogenide semiconductors, or a nanowire array such as semiconducting carbon nanotubes, ZnO, InP based nanowires. Unique alignment procedure of nanowires or nanoribbons along a certain selected orientation, e.g., along the same direction as the electrode pair aligned position, will make the positioning of the nanowires or nanoribbons array onto the electrode pair surface to more easily form a desired bridge array.

In various embodiments, aligned nanowires or nanoribbons obtained from a liquid solution allows for a subsequent transfer of the aligned nanowires or nanoribbons onto a device electrode surface. In various embodiments, the liquid for dispersing randomly oriented nanowires or nanoribbons may be based on water, alcohol, or other solvents or aqueous solvent blends, optionally including additives such as to control the viscosity or to enhance dispersion of the nanowires or nanoribbons in the liquid. The shear-aligned nanowires or nanoribbons may then be picked up and transferred in a group. The transferred nanowires or nanoribbons can optionally anchor as a robust bridge structure, using dielectric or metallic coatings on regions away from the nanobridge location. Processing methods, structures and applications of such a single-bridge nanoribbon or nanowire biosensors are discussed in connection with the various drawings (e.g., FIGS. 19-24).

With reference now to the drawing figures, FIG. 1B(c) shows a MoS₂-based or Si-based, two electrode or gated molecular sensor 15 for genome or DNA sequencing. Such a sensor is configured to detect a change or perturbation in an electrical parameter (e.g., a current pulse) upon the attachment of a nucleotide or other biomolecules to the polymerase 9. In other configurations, the detection event may comprise protein sensing. The MoS₂- or Si-based, two electrode or gated molecular sensor for genome or DNA sequencing 15 is shown to include an optional blocking agent 13 (e.g., PEG, Teflon, etc.) to prevent biomolecule adhesion, and vertical Au or other conducting metal nanopillars 14. The MoS₂- or Si-based, two electrode or gated molecular sensor for genome or DNA sequencing 15 is shown to include a single enzyme molecule 9 (e.g., DNA or RNA polymerase, or other sensors) attached onto a size limited region of the MoS₂ or Si-material bridge, with a biotin-streptavidin complex 10 (with another connecting moiety, e.g., a silane). The sensor 15 is shown with a double-stranded DNA molecule 11 attached to the enzyme 9. Nucleotide monomers 12 (e.g., A, T, C, G) are detected as they participate in the polymerase reaction.

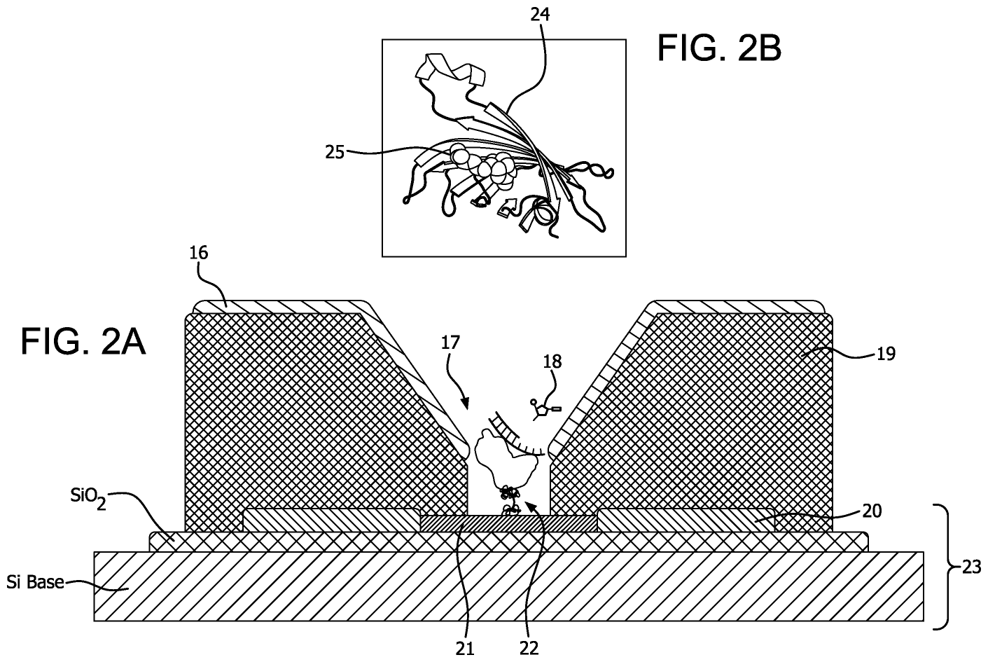

FIGS. 2A and 2B show Design #2 architecture for “Geometrically-guided, single DNA polymerase” molecular sensor on MoS₂ or Si-material bridge (with the exposed area of the TMD or Si-nanoribbon bridge at the base of the channel size restricted to ˜5 nm dia., which is comparable in size to a streptavidin or polymerase molecule). FIG. 2A shows a FET sensor 23 based on TMD (MoS₂ or WS₂) or Si-type nanoribbon bridge for genome or DNA sequencing via detection of change of current pulse upon attaching of nucleotide or other biomolecules. The structure 23 includes a PMMA, HSQ or silica based funnel structure 19 (i.e., a guiding structure), which comprises a tapered channel prepared by e-beam or microfabrication, or by nanoimprint mold (Si or metal). The structure 23 includes a deposited metal electrode pair 20 for source and drain, with an optional third electrode as a gate structure (not illustrated), optional blocking agent 16 (e.g., PEG, Teflon) to prevent unwanted biomolecule adhesion), MoS₂ or Si-material nanoribbon bridge 21, a single polymerase 17 attached onto a size-limited MoS₂ or Si-material nanoribbon bridge, and biotin (on a semiconductor nanoribbon surface, optionally with silane linker) plus streptavidin (on polymerase), or alternatively, an antibody-antigen linkage, or other binding mechanism 22 to connect the polymerase or other enzyme to the TMD or Si-material layer 21. Nucleotide monomer 18 (e.g., A, T, C, G, etc.), fragments, or proteins are to be detected upon polymerase reaction. FIG. 2B shows a polymerase binding mechanism comprising streptavidin 24 and biotin 25.

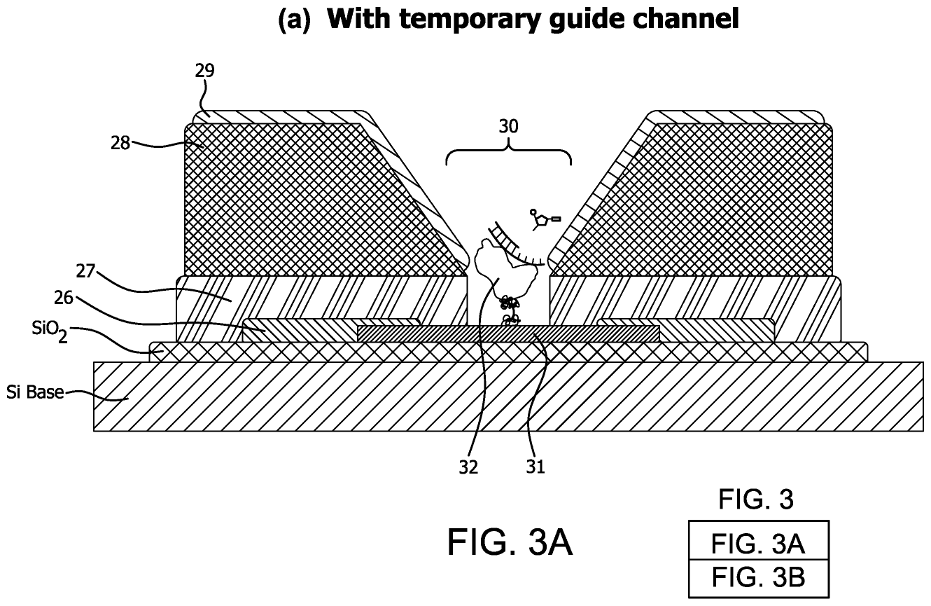

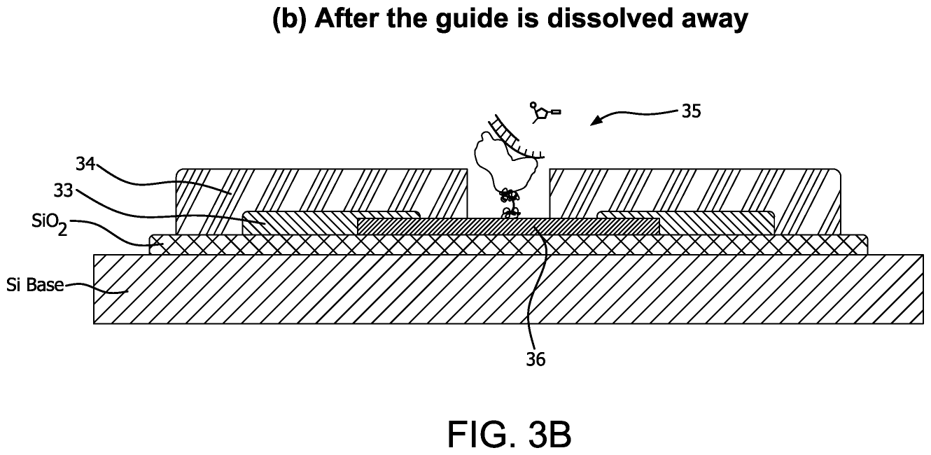

FIGS. 3A and 3B show Design #3 architecture for “Temporary guiding channel to construct single DNA polymerase” molecular sensor on TMD (e.g., MoS₂) or Si-material bridge (with the exposed area at the base of the channel size restricted to ˜5 nm dia., which is comparable in size to a streptavidin or polymerase molecule).

FIG. 3A(a) shows a structure with a temporary guide channel (or guidance channel) in place. The structure optionally includes a temporary and removable (sacrificial) layer 28 (e.g., dextrin, polysaccharide, polyvinyl alcohol, etc.) that can be dissolved in water, alcohol or various other solvents, or dissolvable resist such as PMMA, with optional blocking agent 29 (e.g., PEG, Teflon); an insoluble dielectric 27; deposited metal electrode pair 26 for source and drain, with an optional third electrode as a gate electrode (not illustrated); and TMD (e.g., MoS₂) or Si-type nanoribbon bridge 31. A single polymerase 32 is shown attached to the TMD or Si-material bridge 31.

FIG. 3B(b) shows the device structure after the temporary guide channel is dissolved away. The structure includes a dielectric layer 34 such as SiO₂, cured PMMA, HSQ, etc. This layer is previously disposed completely over the first and second electrodes 33 to entirely encase them, but only partially disposed over the TMD, Si, or doped Si-semiconductor layer 36 so as to leave an opening vertically through the dielectric layer to an exposed portion of the TMD, Si, or doped Si-semiconductor layer. As shown in FIG. 3B(b), the exposed portion has a diameter less than the width of the nanogap between the previously deposited metal electrodes 33. In this way, the nanogap between electrodes 33 does not need to be precisely manufactured since it will be the dielectric layer 34 that determines the size of the opening to accommodate the biomolecule. As shown, the structure includes a single polymerase sensor complex 35 partially hidden below the dielectric layer 34, attached to the TMD, Si, or doped Si-semiconductor layer 36 by such linkers as streptavidin-biotin linkage.

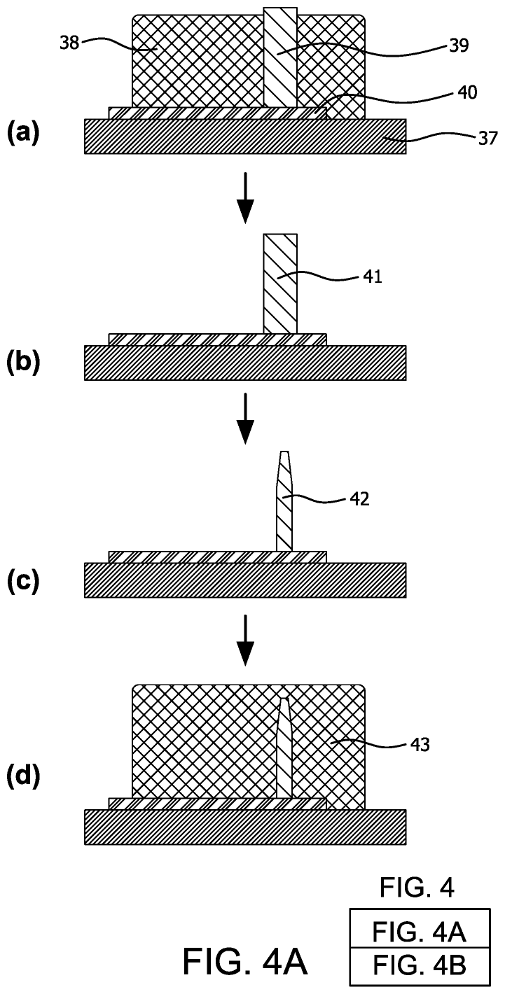

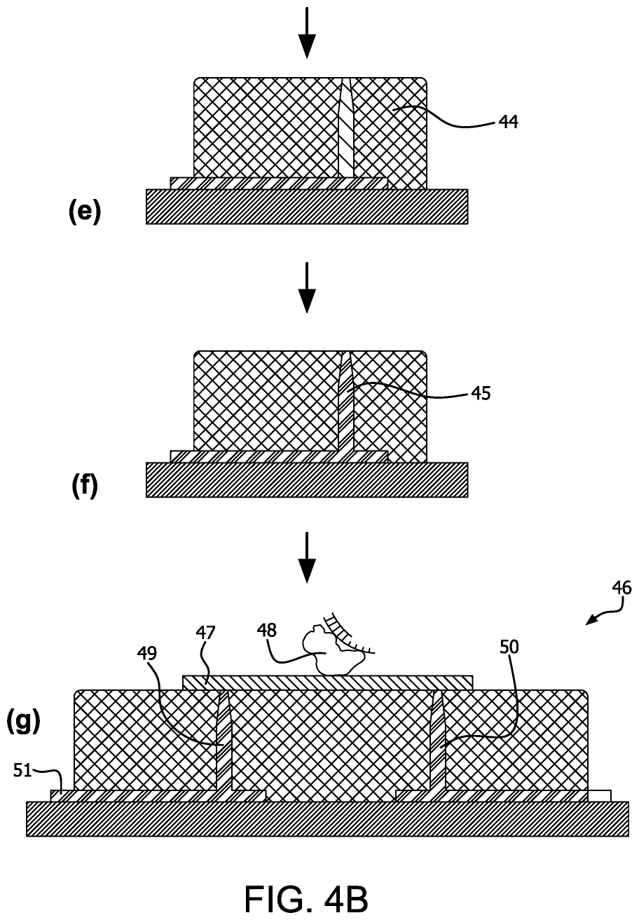

FIGS. 4A and 4B show a fabrication method designed to provide narrowed pillar structures that then can be used in a sensor device. In FIG. 4A(a), the structure includes Au or other conducting lead wire 40 on a substrate 37. E-beam or nanoimprint lithography may be used to configure a vertical hole in the PMMA 38 down to the surface of the conducting layer 40. The resulting hole is then filled with Cu or Ni, such as by electroless or electrodeposition, or a sputter and lift-off process, to produce a Cu or Ni nanopillar 39. In FIG. 4A(b), the PMMA is dissolved away to expose the bare Cu or Ni nanopillar 41 (which may be about 20-50 nm in dia.). In FIG. 4A(c), chemical or RIE etch is used on the Cu or Ni nanopillar to form a nanowire 42 with reduced diameter and tapered tip (e.g., with a tip ending in a diameter of ˜5 nm). In FIG. 4A(d), PMMA 43 is cast over the Cu or Ni nanowire 42 to cover the diameter-reduced Cu or Ni nanowire. In FIG. 4B(e), the PMMA 44 is planarized in height by RIE to expose the Cu or Ni nanopillar top. In FIG. 4B(f), the Cu or Ni nanopillar is dissolved away to create a hole, and Au or other metal is electrodeposited to form the new pillar 45 (having only ˜5 nm dia. tip area). This narrowed nanopillar 45 can then size-limit the MoS₂ or Si-material width or the number of any attachable nanowire/nano-ribbon bridge. FIG. 4B(g) shows a complete molecular bridge solid state sensor 46 that is configured to detect a change of current, such as a perturbation or pulse, or other signals upon attaching or detaching of nucleotides or other biomolecules. Molecular bridge solid state sensor 46 includes: Au or other conducting lead wire 51 disposed on the underlying substrate; Au nanopillars 49 and 50; MoS₂ or Si-type nanobridge 47; and single enzyme (e.g., DNA or RNA polymerase) 48 attached onto the MoS₂ or Si-material nanobridge 47. In this way, the TMD or Si-material nanobridge 47 is elevated above the gap between electrodes 51 by the height of the pillars 49/50.

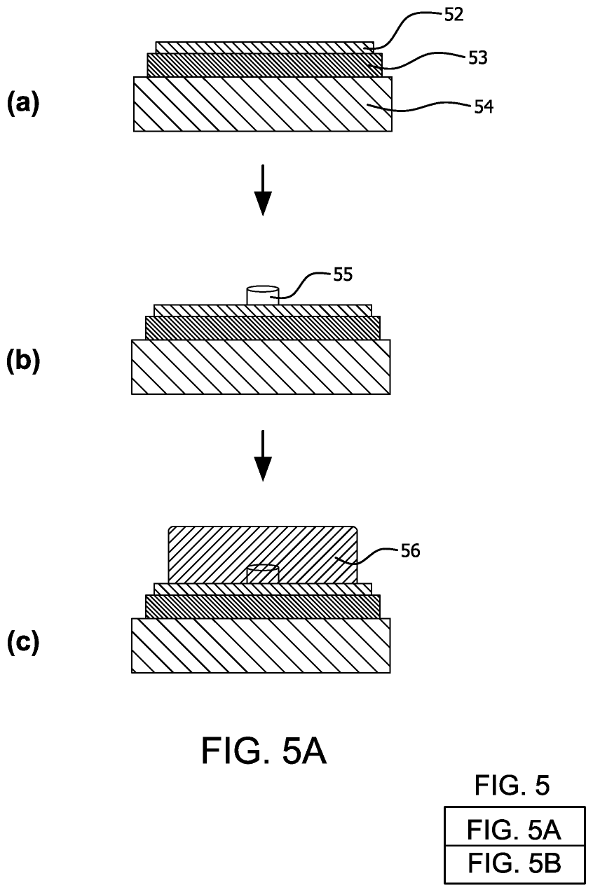

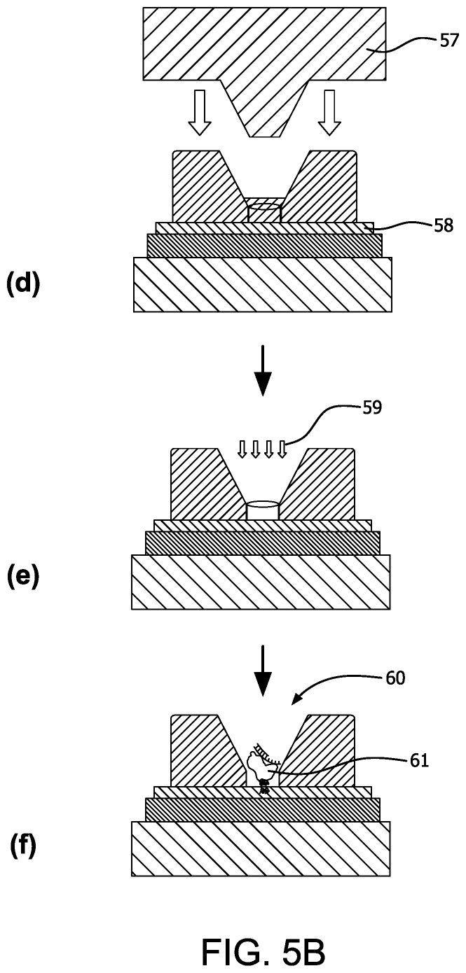

FIGS. 5A and 5B show another sensor fabrication method. In FIG. 5A(a), the structure (a) comprises a MoS₂ or Si-material nanoribbon 52 disposed on an underlayer 53 (e.g., PMMA, SiO₂), which is disposed on a substrate 54 (e.g., Si). In FIG. 5A(b), a dissolvable pillar 55 (5-10 nm dia., comprising Cu, Ni, etc.) is deposited by e-beam or NIL lithography onto the TMD or Si-material nanoribbon. In FIG. 5A(c), a resist 56 (e.g., PMMA, HSQ, or another type of silica-containing resist) is spin coated over the entirety of the pillar 55. In FIG. 5B(d), an imprint is made with a die 57 (e.g., the die 57 made of Si, SiO₂, or metal alloy mold). The MoS₂ or Si-material nanoribbon 58 remains unchanged throughout the process. In FIG. 5B(e), the metal pillar is exposed by RIE etching 59 (the directional arrows indicating the etching process to clean off the pillar). In FIG. 5B(f), the metal pillar is dissolved away to create a funnel shaped guiding structure 60 with e.g., a 5-10 nm hole, so that only a single streptavidin molecule or only a single polymerase or other enzyme can be placed therein for more accurate sequencing by the finished device. The structure in FIG. 5B(f) includes a single polymerase 61 attached onto a size-limited MoS₂ or Si-material bridge (by e.g., by biotin-streptavidin linkage or other linkage).

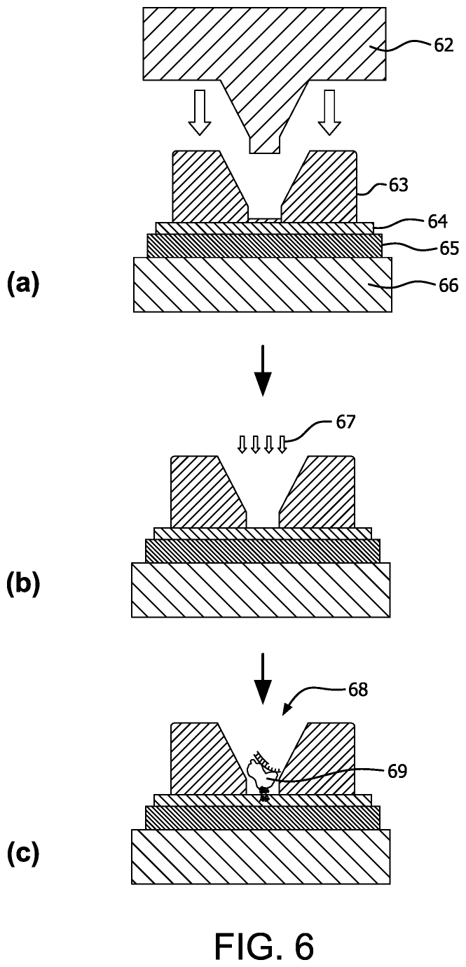

FIG. 6 shows another method of fabricating a sensor device. In FIG. 6(a), the structure (a) comprises a MoS₂ or Si-material nanoribbon 64 on a PMMA layer 65, which is on a Si substrate 66. A PMMA or HSQ (which can be later converted to SiO₂) thermo-plastic resist or other silica-containing resist 63 is imprinted with an appropriately shaped die 62 (e.g., made of Si, SiO₂, or metal alloy mold) having a <10 nm dia. tip protrusion as illustrated. In FIG. 6(b), RIE etch 67 exposes the base MoS₂ or Si-material bridge nanoribbon surface. In FIG. 6(c), through the funnel shaped guiding structure 68 with e.g., 5-10 nm hole is placed only a single streptavidin or only a single polymerase or other enzyme for more accurate sequencing from the finished sensor device. FIG. 6(c) shows a single polymerase 69 attached onto a size-limited MoS₂ or Si-material bridge (by e.g., a biotin-streptavidin linkage or other linkage).

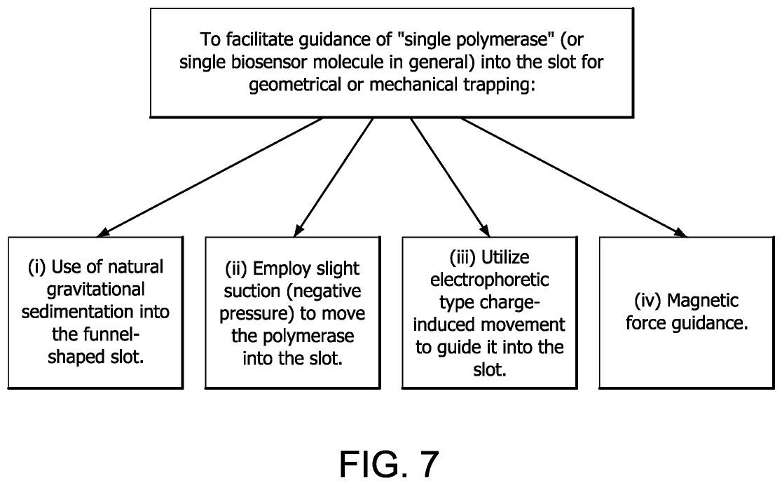

FIG. 7 shows how to facilitate guidance of “single polymerase” (or a single biosensor molecule in general) into the slot for geometrical or mechanical trapping: (i) use of natural gravitational sedimentation into the funnel-shaped slot; (ii) employment of a slight suction (negative pressure) to move the polymerase into the slot; (iii) utilizing electrophoretic type charge-induced movement to guide the molecule into the slot; or (iv) magnetic force for guidance.

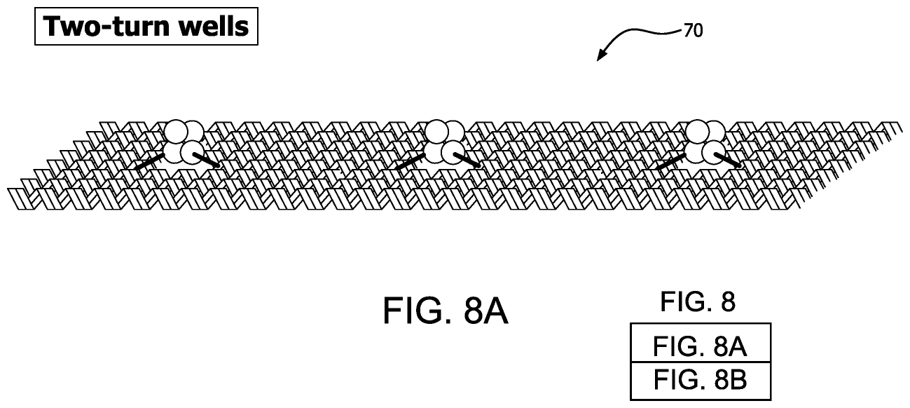

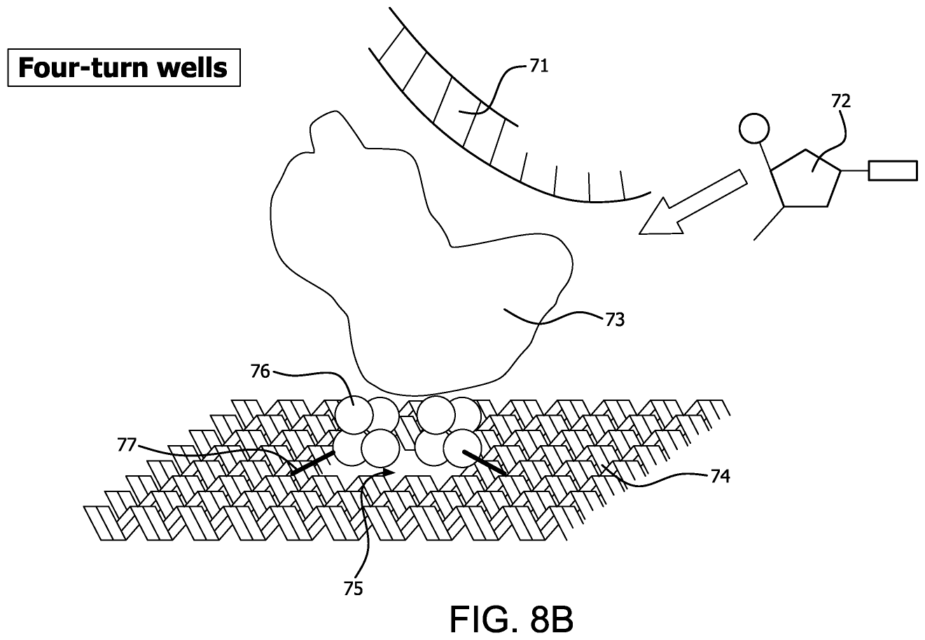

FIGS. 8A and 8B show Design #4 “Use of size-confined DNA assembly well”: size-confined nano-well by DNA assembly on MoS₂ or Si-type nanoribbon bridge; periodic array of streptavidin (SA) on electrode surface, or such a template further processed/coated (with dielectric or conductive layer) for geometrical size-limiting constraint to enable only (or essentially only) a single enzyme molecule attachment; SA nanoarray is prepared by size-selective capturing of a single SA tetramer in a two-turn well (FIG. 8A, 70) or capturing of two tetramers trapped in a four-turn well (FIG. 8B).

As illustrated in FIG. 8A, two-turn wells 70 (20 nm-10 μm) spaced apart are used for each electrode pair in a multi-sensor array. In FIG. 8B, a four-turn well 75 (˜7×10 nm well) in a DNA bundle 74 has trapped streptavidin 76 (two streptavidin tetramers), and biotin 77 linking. FIG. 8B shows double strand DNA 71, nucleotide monomer 72 (A, T, C, G, etc.), short fragments, proteins to be detected, and preferably a single enzyme molecule 73 (e.g., DNA or RNA polymerase) attached onto size limited TMD bridge like MoS₂ or WS₂ or a Si-material bridge.

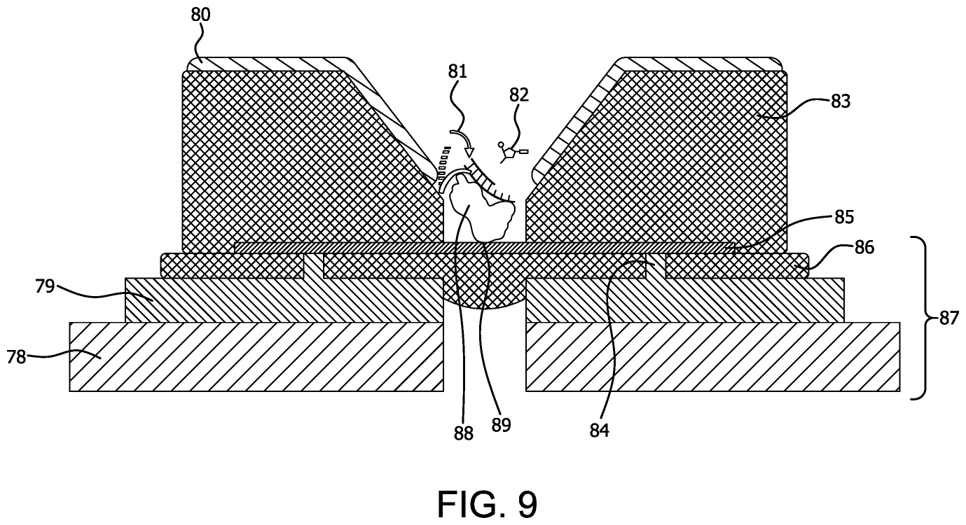

FIG. 9 shows Design #5“Use of physically-trapped single polymerase on TMD (MoS₂) or Si-material bridge on vertical nanopillar conductor.” Such a device design reduces contact resistance and further increases the FET signals, (e.g., by at least 10 times), and eliminates the need for biotin and streptavidin type linkages since the enzyme may be guided into the device to attach directly to the TMD, Si- or Si-semiconductor layer 85. The sensor device uses shape memory polymer or an alloy structure to physically force electrical contact (Van der Waals force bond) of a polymerase molecule onto the TMD (MoS₂) or Si-type bridge. In FIG. 9, the molecular sensor on the TMD or Si-material bridge 87, usable for genome or DNA sequencing via detection of change of current pulse upon attaching of nucleotide or other biomolecules, comprises Au or other conducting metal leads 79 disposed on a dielectric substrate 78 (SiO₂, etc., on Si). Electrically connected to the metal leads 79 are vertical Au (or other conducting metal) nanopillars 84, which can be formed, for example, by the method embodied in FIGS. 4A-4B and discussed above. In this structure, the PMMA or other dielectric material layer 86 is planarized such that the top surface of the PMMA layer 86 is entirely planar with the exposed tips of the nanopillars 84. In this way, the exposed tops of the nanopillars 84 will appear as discs. The planarized PMMA 86 layer enables deposition or transfer of the TMD, Si- or Si-semiconductor layer 85 on the device with guaranteed contact to the tops of the two nanopillars 84. The PMMA channel 83 is used to direct the enzyme or enzyme complex into the exposed region of the TMD, Si- or Si-semiconductor layer 85.

In FIG. 9, the conducting electrodes 79 are separated by a nanogap having a width W1. This width may be from about 2 nm to about 100 nm, and more preferably about 5 nm to about 20 nm. The pair of nanopillars 84 are separated from one another by a width W2, wherein in various embodiments W2≥W1. In various embodiments, W2 may be from about 5 nm to about 100 nm. With the TMD, Si- or Si-semiconductor layer 85 attached across and connecting to the tops of the nanopillars 84, the TMD, Si- or Si-semiconductor layer 85 is suspended over the space between the nanopillars 84 and the over the nanogap. Since the former space is filled in with dielectric material 86, that material may partially or entirely fill in the nanogap between the electrodes 79. As shown in the example of FIG. 9, the dielectric material entirely fills in the space between the nanopillars 84 but only partially fills in the nanogap.

In FIG. 9, blocking agent 80 (e.g., PEG, Teflon) may be used to prevent unwanted biomolecule adhesion to areas other than the exposed portion of the TMD or Si-material bridge 85 below. Shape memory change 81 enables fixing a single polymerase 88 directly to this size-limited TMD or Si-material bridge, with nucleotide monomer 82 (e.g., A, T, C, G, U, etc.) to be detected on polymerase reaction. In FIG. 9, the linkage 89 between enzyme 89 and TMD or Si-material bridge 85 may comprise biotin-streptavidin, or an antibody-antigen linkage, or other linkage mechanism, or the connection may comprise a bond between a modified amino acid of the enzyme and the transition metal or silicon of layer 85.

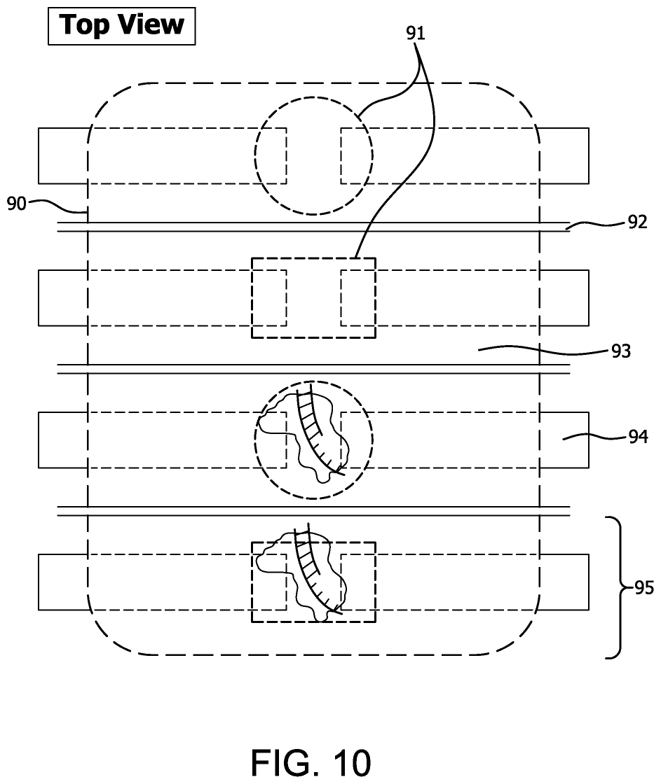

With reference to FIG. 10, a TMD (MoS₂) or Si-material sheet 90 is configured with size-limited (circular, square, or other desired shapes) areas 91 to locally and selectively expose the TMD or Si-material. A large sheet can be placed by lifting up of floating sheet onto device surface covering many electrode pairs lithography-patterned, FIB separated, or mask patterned. Optional FIB (focused ion beam) slicing, e-beam/NIL slicing 92 of MoS₂ or Si-material can be used to separate portions from adjacent devices. The exposed island region 91 is surrounded by pattern defined dielectric coating mask 93 (e.g., PMMA, PDMS, other adhesion blocking PEG type layer or Teflon, etc.) to prevent/minimize biomolecule attachment. An array of conducting electrode and lead wires 94 are used for signal detection. In FIG. 10, one complete molecular bridge sensor 95 is illustrated and comprises a MoS₂, WS₂ or Si-material layer bridge.

In various embodiments, the array of electrode pairs in FIG. 10 may further comprise nanoribbon or nanowire bridges, wherein a single bridge is disposed across two electrodes in a single pair of electrodes. In this way, the size-limited (circular, square or other shape) regions provide only limited exposed regions of the nanoribbon or nanowire bridge on each pair of electrodes. In various embodiments, each nanoribbon or nanowire bridge on each pair of electrodes may comprise, for example, graphene, SWCNTs, MoS₂, DNA, or polypeptides. These nanoribbon or nanowire bridges may have been pre-aligned into parallel arrays before transfer over the top of the array of electrode pairs.

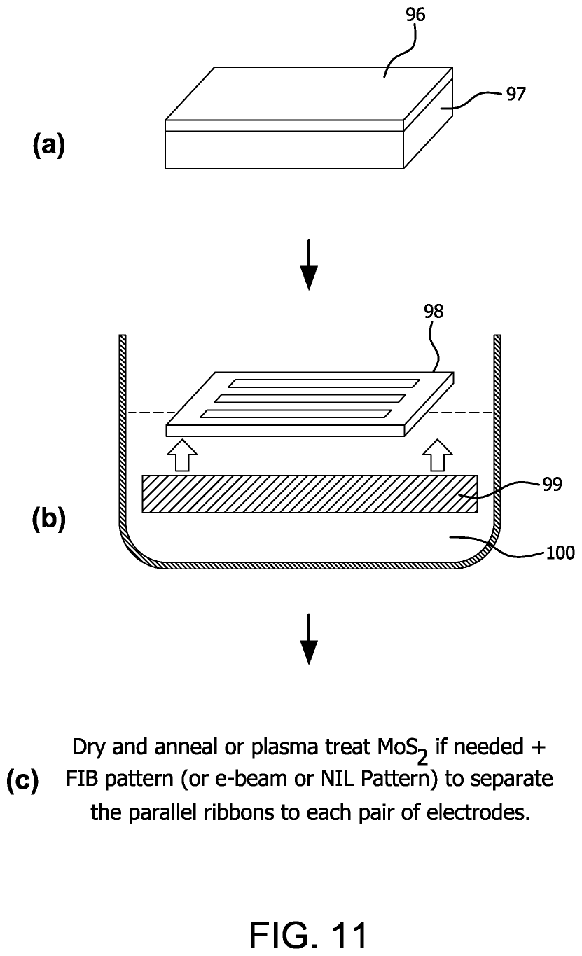

FIG. 11 shows a “Wet transfer of un-patterned or pre-patterned MoS₂ ribbon array: MoS₂ films are synthetized at >700° C., so they are preferably pre-made and then transferred onto a sequencing device. The next steps in the process are to (i) release the ribbon array in an aqueous solution (chemically dissolve away the substrate); (ii) place the Au electrode device (wafer) in the solution; (iii) lift up the wafer to catch the floating MoS₂ film; (iv) dry and anneal the wafer to strongly bond the MoS₂ film (or nano-ribbon) onto the electrode surface; and (v) prepare size-limited MoS₂ regions and attach DNA polymerase. The finished sensor functions by generating detectable sequence signals corresponding to nucleotide attachment to the polymerase.

In FIG. 11(a), the substructure (a) may be prepared by synthesis of TMD 96 like MoS₂ or WS₂ (e.g., by exfoliation, CVD or sulfurization) on Si or metal substrate 97. Substrate 97 can comprise Si with SiO₂, a SOI wafer or a metal layer. In the transition from (a) to (b), substrate is etched away for floating MoS₂.

In FIG. 11(b), floating MoS₂ or WS₂ 98 (no pattern or pre-pattered into parallel ribbons, still attached) in H₂O or alcohol 100, with SiO₂-coated Si substrate 99 (optionally with Au electrode pair disposed on it), is lifted up to pick up the MoS₂ layer.

In FIG. 11(c), the wafer is dried and annealed or plasma treated if needed, and optionally FIB patterned (or e-beam or NIL patterned) to separate the parallel ribbons such that one ribbon is associated to each pair of electrodes.

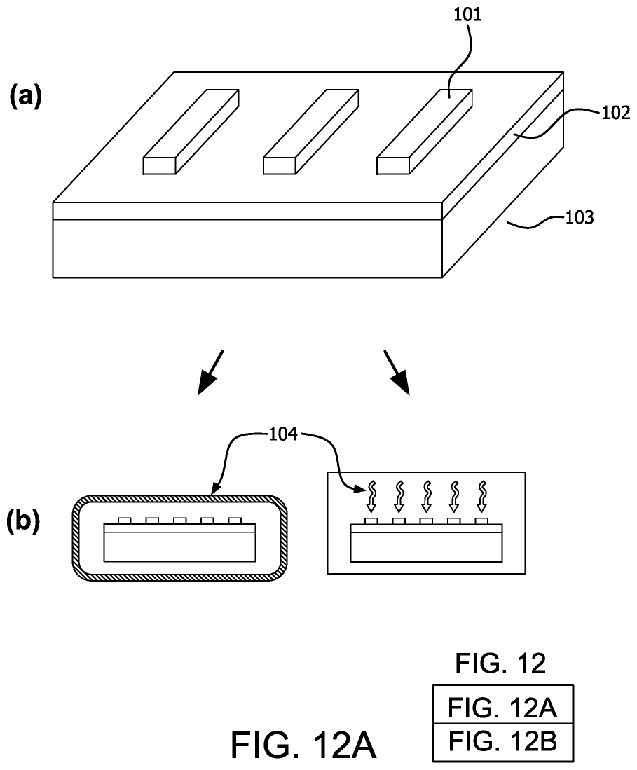

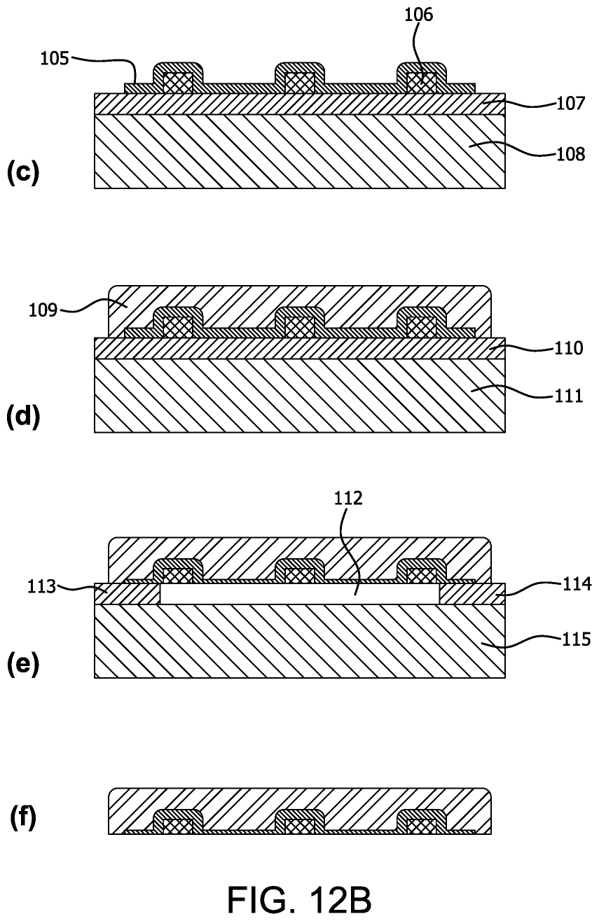

FIGS. 12A and 12B illustrate various aspects of a fabrication method for parallel nanoribbons. The structure in FIG. 12A(a) comprises a nano-patterned MoS₂ or Si-material nanoribbon array 101 disposed on SiO₂ 102, which is disposed on Si base 103.

In FIG. 12A(b), high-temp annealing or plasma treatment 104 can be used to repair any possible damages on the patterning.

In FIG. 12B(c), nano-patterned MoS₂ or Si-material nanoribbon array 106 on SiO₂ 107 on Si base 108 is optionally coated with a removable polymer 105 (e.g., dextrin, glucose, grease, wax).

In FIG. 12B(d), the nano-patterned MoS₂ or Si-material nanoribbon array on SiO₂ 110 on Si base 111, optionally coated with removable polymer (e.g., dextrin, glucose, grease, wax), is then cast over with a PMMA or PDMS elastomer 109, and then cured.

In FIG. 12B(e), the SiO₂ 112 underneath is etched away through intentionally added slots in the PMMA or PDMS to release the potted nanoribbon array. This can also be done in two steps of PMMA followed by PDMS. In FIG. 12B(e), the structure shown comprises portions of SiO₂ 113 and 114 after etching, on the Si base 115.

FIG. 12B(f) shows the released material that can be washed lightly to make the MoS₂ or Si-material nanoribbons protrude slightly, dried, and then transferred and pressed onto the sequencing device to be released, and form a non-bridge between two mating metallic electrodes.

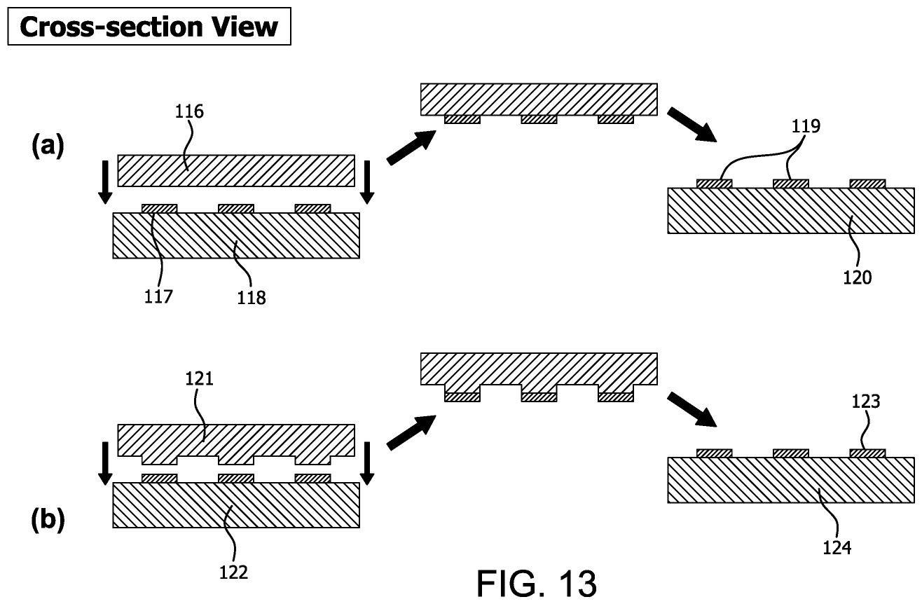

FIG. 13 shows another fabrication method in accordance with the present disclosure. FIG. 13(a) shows a PDMS stamp 116 (with a flat contact surface), and MoS₂ nano-ribbon array 117 (nanopatterned on a flat substrate 118). The transformation illustrated comprises a pickup of the nanoribbons by stamp, and a release of the nano-ribbons 119 on a device surface 120 (e.g., as a bridge between two mating electrodes). In FIG. 13(b) a pre-shaped PDMS stamp 121 is used to pick up the nanoribbons from substrate 122 by stamp, and then the nanoribbons 123 are released on the device surface 124 (e.g., as a bridge between two mating electrodes).

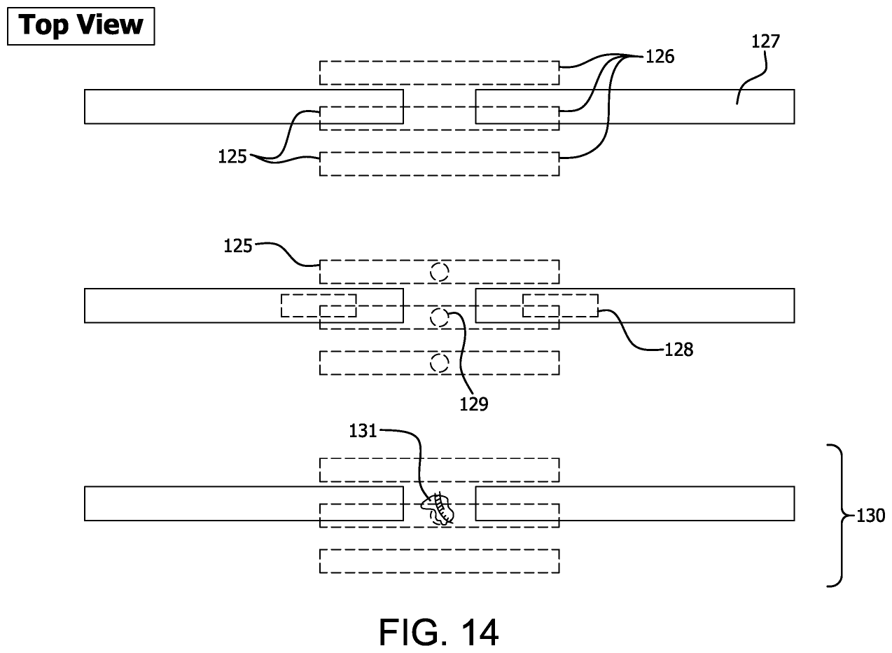

FIG. 14 shows MoS₂ or Si-material nanoribbons 125 in a pre-patterned parallel array 126 of MoS₂ or Si or doped silicon with redundant ribbon array (made by e.g., e-beam lithography, nanoimprint lithography, template-assisted nanopatterning, etc.). The imprint may be transferred onto an electrode array so as to increase the probability of MoS₂ or Si-bridge connection. The MoS₂ or other TMD or Si-material ribbons can be Van der Waals force attached, or optionally metallization deposit (metallization deposit 128 (Ti, Au, Ni, etc.) over MoS₂ or Si-material can be made to more firmly attach the ribbons on the electrodes. In FIG. 14, optional addition of local biotin-binding-enhancing coating 129, or surrounding-area-masking can be employed to limit the exposed MoS₂ or Si-material regions to ˜5-10 nm dia. One complete molecular sensor 130 is illustrated that comprises a MoS₂ or Si-material bridge with a polymerase assembly, shown with a polymerase enzyme 131 (with associated biotin-streptavidin or other type linkage).

FIG. 15 shows another fabrication method in accordance with the present disclosure. In FIG. 15(a), a tethered array of encoded (memory written) DNA fragments 132 is periodically positioned on a substrate. Molecular nanobridges (MoS₂ or Si-material) polymerase sensor array 133 (w/o DNA template). Magnified view shows polymerase 134, linker 135, and Au electrode pair 136. In FIG. 15(b), polymerase-MoS₂ or Si-type nanobridge array approaching DNA array being released (indicated by 137). In FIG. 15(c), nanobridge sensor array polymerase molecule array picks up DNA templates and is moved away (indicated by 138). Magnified view shows DNA template 139, polymerase 140, linker 141 and Au electrode 142.

In various embodiments, FIG. 15 illustrates an exemplary DNA memory device utilizing a sequencing analysis biosensor bridge structure. FIG. 15(a) depicts a tethered array of encoded (memory written) DNA fragments 132 periodically positioned on a DNA memory reader 133 comprising transfer anchored nanoelectrode bridge and polymerase sensor array. FIG. 15(b) depicts transferred and anchored nanobridge array (or other semiconductor nanobridge array) approaching an information stored DNA array, and then being released as indicated by the upward arrows. FIG. 15(c) depicts DNA templates taken up by a polymerase array and how the completed sensor array is moved away for DNA sequencing to read the recorded memory information. In this figure, a microfluidics chamber is not shown. FIG. 15 illustrates how in various embodiments a massively parallel DNA written information array in combination with a massively parallel nanoribbon bridge reader array allows random-access-enabled DNA memory retrieval. Several different configurations of tethered DNA memory arrays having various methods of tethering and releasing, and sensor access schemes, can be made to maximize memory processing capability.

FIG. 16 illustrates a fabrication method in accordance with various embodiments of the present disclosure. The method begins with a silicon-on-insulator (SOI) wafer 160, shown in (a), comprising a Si-material layer 161 disposed on a dielectric substrate layer 162, e.g., SiO₂, which is disposed on the Si base 163 of the SOI wafer. As discussed throughout, the Si-material top layer may comprise crystalline Si, such as single crystal Si, or a p-type or n-type doped Si. This Si material layer 161 may have a thickness of less than about 50 nm. In various embodiments, the thickness of the Si-material layer 161 is less than about 20 nm. The Si-material layer may comprise various n-doped or p-doped semiconductor material, such as Si, Ga—As, In—P, ZnO based, or other thin layer semiconductor materials such as graphene bases, 2D semiconductor-based (e.g., MoS₂, WS₂ or other chalcogenide based semiconductors. While doped semiconductors are preferred, if the nanoribbon width is narrowed sufficiently per the patterning described below, there is a possibility of bandgap opening, so undoped or minimally-doped semiconductors may also be utilized. Such SOI wafers are commercially available with the type of semiconductor material 161 as desired.

With continued reference to FIG. 16, the next step in the fabrication is to pattern the Si-material layer 161 into a pattern of parallel Si-material nanoribbons 164. This patterning may be accomplished by e-beam lithography, nanoimprint lithography, graphoepitaxy, shadow-mask RIE etching, or by various other methods of patterning. The resulting structure (b) comprises parallel Si-material nanoribbons disposed on the dielectric substrate layer 162 of the SOI wafer. The nanoribbons 164 can of course by patterned into more of rod shapes, or other cross-sectional shapes as needed for particular sensor structures. In various embodiments, the width of each ribbon or rod is less than about 30 nm, preferably less than about 10 nm, and most preferably less than about 6 nm. In an alternative to manipulating an SOI wafer, an array of parallel semiconductor nanoribbons or rods can be fabricated on a different substrate surface, and then taken off the surface (e.g., using PDMS or adhesive polymer stamps) and transferred onto a device surface. Some adhesion-enhancing layers that can be etch-removed later (e.g., Au, Ag, Cu, Ni, etc.) can be optionally added as the surface contacting the semiconductor nanoribbon to pick them up more easily for transfer onto the device substrate surface.

With reference now to structure (c) in FIG. 16, pairs of electrodes 165 may be deposited onto the array of parallel nanoribbons 164 such that two electrodes in any single pair of electrodes contacts either side of a single nanoribbon as illustrated. A partially finished sensor unit is illustrated as the second assembly from the right of the structure, comprising a biotin-streptavidin complex 166, with optional linker such as a silane, attached to the nanoribbon 164. A finished sensor unit is shown at the far right of the structure, comprising a sensor complex 167 further comprising a single molecule enzyme, such as a DNA or RNA polymerase, attached to the Si-material nanoribbon bridge, with a DNA or RNA template attached and ready for nucleotide interaction and detection. The wafer structure can be encased in a fluidic chamber such that the appropriate biomolecules can be circulated over the exposed nanoribbons for attachment onto the nanoribbons. A complete sensor having the sensor complex works by providing changes in an electrical property of the Si nanoribbon FET bridge, such as current perturbations, when nucleotides attach to the polymerase and participate in polymerase reactions.

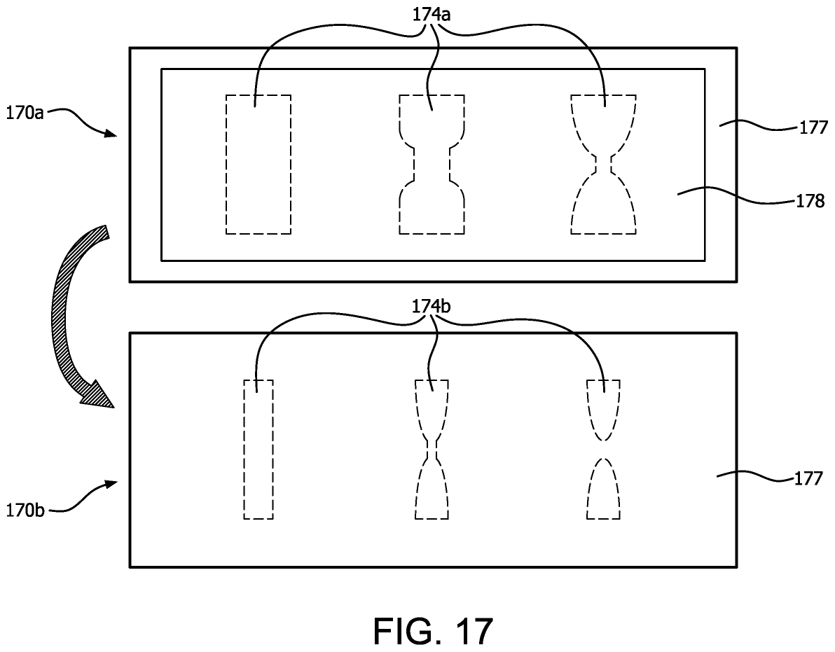

FIG. 17 illustrates various dimensions and shapes for semiconductor nanobridges, which are controllable by nanofabrication, nanoimprinting, shadow-mask RIE etch or by repeated oxidation/chemical etch. In structure 170a at the top of FIG. 17, different shaped and/or sized nanoimprinted, nanopatterned, shadow-mask RIE etched Si-material, (Si or a doped Si semiconductor), TMD material such as MoS₂, graphene or an amorphous semiconductor nanoribbons 174a, are disposed on a thin Si semiconductor or n- or p-type Si semiconductor layer 178, which is disposed on the substrate 177 of an SOI wafer. Alternatively, the structure of layers 178 and 177 may be another type of dielectric covered substrate instead of an SOI wafer. Structure 170a is converted to structure 170b by further narrowing the Si or other semiconductor nanoribbon array. This width reduction can be accomplished by nanopatterning or repeated oxidation and chemical etching. The resulting width-reduced nanoribbons 174b are left disposed on the underlying SOI wafer substrate 177. Having reduced nanoribbon width (i) opens a bandgap; (ii) allows higher signal-to-noise ratio electrical signals; and (iii) enables a single or just a few streptavidin or DNA molecules to attach to any single nanobridge surface. If a nanoribbon tip is sharpened such that the nanoribbon separates into portions, as per the nanoribbon illustrated at the far right, electric field concentration of the sharp-tip electrode can allow a single DNA or other type of biomolecular bridge to form by dielectrophoretic (DEP) type or electrical field alignment. This sharpening of a nanobridge as shown to produce a two-portion semiconductor can be accomplished by further etching or by FIB slicing.

FIG. 18 illustrates a method of alignment and attachment of floating nanoribbons 198a onto electrode pairs 185a, such as arranged in an array of electrode pairs. The structure (a) is before any electric field is applied and structure (b) is after electric field alignment of the nanobridges to the electrode pairs. In this method, the floating nanoribbons 198a may comprise Si nanoribbons, graphene, metal dichalcogenide, such as MoS₂ type nanoribbons, amorphous semiconductor nanoribbons, any of which may be pre-patterned or pre-narrowed. For the method, the electrode pairs are encased in a fluidic chamber 190, like a flow cell. The block arrows 191 indicate the flow of the microfluidic solution moving. To attach the nanoribbons to the electrode pairs, dielectrophoretic (DEP) alignment is used, whereby an AC or DC electric field is applied to the electrode pair array. The applied AC field may be in the range of about 100 mV to about 20 V, and preferably from about 50 mV to about 5V, with a frequency of about 50 Hz to about 10 MHz, and preferably from about 100 Hz to about 100 KHz. The result after electric field alignment is shown in structure (b), wherein nanobridges 189b are now attached to electrode pairs 185b, with one nanobridge per electrode pair. As illustrated, there is an excess of nanobridges and thus some do not participate in attachment.

FIG. 19 illustrates various embodiments of a method of obtaining a plurality of parallel aligned nanowires and nanoribbons that can be later transferred in groups. The nanowires and nanoribbons may comprise such materials as TMD (MoS₂, WS₂, etc.), Si or doped silicon semiconductor materials, graphene, carbon nanotubes (CNT, SWCNTs), ZnO, InP, GaAs, or various other semiconductors. The method begins with a structure 200 shown in (a), which comprises a quartz or other crystal wafer 201 configured with a plurality of crystallographic steps 202a. The number of steps 202a may correspond to the number of parallel nanoribbons or nanowires desired, or may provide groups of parallel ribbons or wires that contribute in part to an entire array of device structures. The substrate 201 may comprise ST-cut quartz or other crystals. Each step 202a further includes a corresponding corner 202b, such as one might see on a macroscopic scale when a vertical step plate meets the horizontal step. The crystal 201 may be cut to a selected orientation, such as cut at 42.5° angles, to create the crystal steps.

As shown in FIG. 19(b), nanoribbons 205a are grown on the crystallographic steps 202a, such as by CVD, vapor crystallization, or chemical synthesis (e.g., low or room temperature). In this way, the crystallographic steps act as the nucleation sites to provide for the pre-aligned nanowires and nanoribbons, with the end result structure 203 having a plurality of nanoribbons 205a on the steps. Other nanowires, such as DNA, polypeptides or their associated structures, may be formed on the step edge sites by near-room-temperature deposition.

As shown in FIG. 19(c), nanowires 205b can be grown in the step corners 202b to produce the structure 204 having pre-aligned nanowires. In various embodiments, CVD can be used to grow a plurality of single-walled CNTs in the corners of the crystal steps. In other embodiments, high temperature annealing, or near-room-temperature chemical synthesis, can produce ZnO or other nanowires in the step corners.

With continued reference to FIG. 19, the resulting plurality of aligned nanoribbons 205a or nanowires 205b can be picked up and transferred in groups onto a surface of a device structure by a PDMS or epoxy stamp, wherein the device surface receiving the transferred nanoribbons or nanowires may optionally already have a metal layer deposit or electrode pairs in a parallel array. In various embodiments of the method, semiconductor nanoribbons or nanowires can have thickness, width or diameter of <30 nm or <20 nm, preferably <10 nm, and more preferably <6 nm. In various embodiments, single wall carbon nanotubes (SWCNTs) provided by the method of FIG. 19 have diameters typically in the range of from about 0.8 nm to about 1.3 nm.

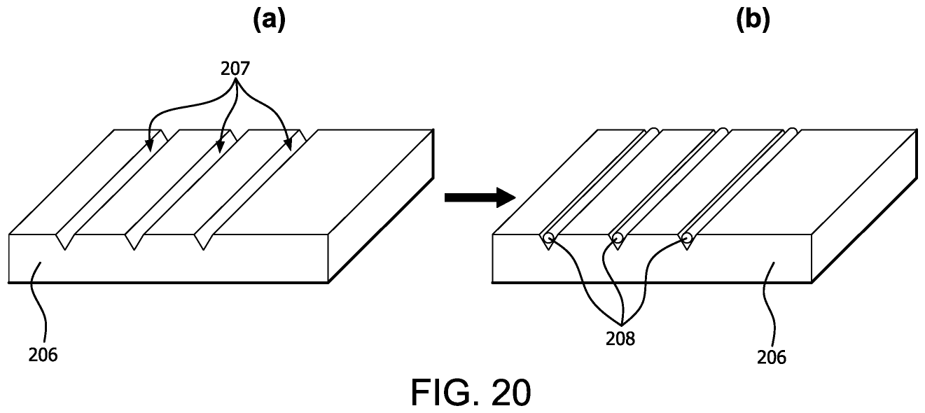

With reference now to FIG. 20, substrate 206 is used for nano-groove induced alignment of nanowires or nanoribbons. The substrate 206 is configured with parallel grooves 207 having nanoscale dimension. The grooved substrate 206 may comprise, for example, Si, sapphire, SiO₂-on-Si, any other crystal or fused quartz. The parallel plurality of grooves may be formed, for example, by chemical etching or nanopatterning.

As illustrated in FIG. 20, structure (a) to (b), the grooved substrate 206 can collect randomly oriented and dispersed nanoribbons or nanowires 208, or can be used as a substrate for growing a plurality of parallel nanoribbons or nanowires. For example, nanoribbons or nanowires 208 may be formed in the grooves by thermal, CVD, chemical synthesis, or the grooved substrate 206 may be used for near-room temperature mechanical alignment of loose nanowires or nanoribbons dispersed in a liquid (e.g., ethanol or water). In this method of nanogroove induced alignment, the nanowires or nanoribbons may include, but are not limited to, CNTs, Si, Si semiconductor materials, ZnO, InP, GaAs, DNA or other oligonucleotides, polypeptides. In various embodiments, high-temperature synthesis of semiconductor nanowires at the grooved sites results in this array of aligned nanowires.

With continued reference to FIG. 20, the resulting plurality of groove aligned nanoribbons or nanowires 208 can be picked up and transferred in groups onto a surface of a device structure by a PDMS or epoxy stamp, wherein the device surface may optionally already have a metal layer deposit or electrode pairs in a parallel array. In various embodiments of the method, semiconductor nanoribbons or nanowires aligned or grown in the grooves can have thickness, width or diameter of <30 nm or <20 nm, preferably <10 nm, and more preferably <6 nm. In various embodiments, single wall carbon nanotubes (SWCNTs) provided by the method of FIG. 20 have diameters typically in the range of from about 0.8 nm to about 1.3 nm.

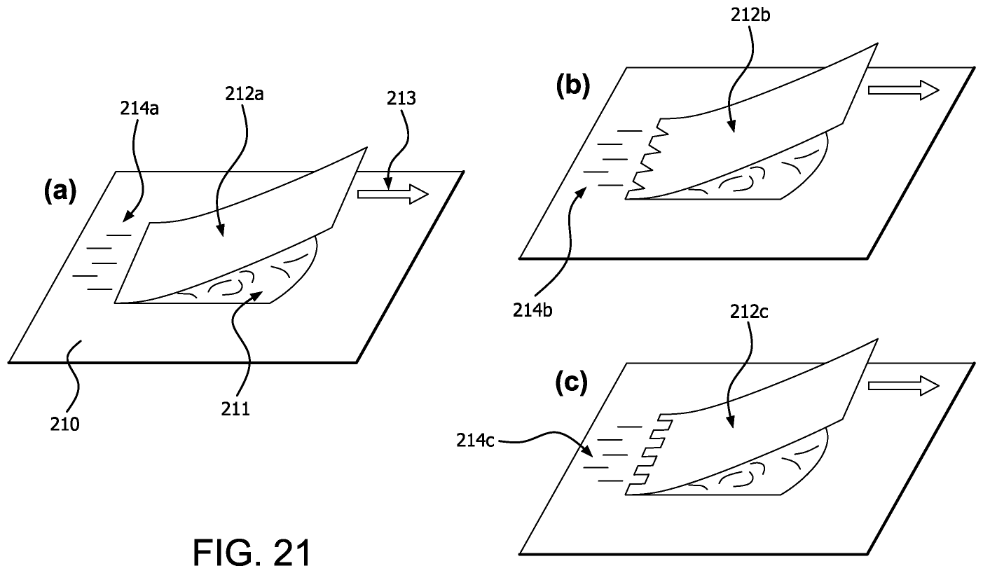

FIG. 21 illustrates various embodiments of a shear-alignment method that provides parallel aligned nanowires or nanoribbons. The method depicted in FIG. 20(a) comprises a substrate 210 with a liquid dispersion 211 of randomly aligned nanoribbons or nanowires thereon. A scraper 212a, having a flat and linear edge against the substrate 210 is dragged through the liquid dispersion 211 in the direction 213 to provide parallel aligned nanowires or nanoribbons 214a. Methods depicted in FIG. 20(b) and FIG. 20(c) are variations where the scraper is configured with a sawtooth edge (scraper 212b) or a notched edge (scraper 212c), respectively. Each of these three scrapers 212a, 212b and 212c, provide shear-aligned nanowires or nanoribbons 214a, 214b and 214c, respectively, from a randomly oriented dispersion 211 of nanowires or nanoribbons.

With continued reference to FIG. 21, the type of nanowires or nanoribbons to be aligned in this method include, for example, (i) organic elongated elements like DNA, peptides or assembled DNAs, or peptide-modified DNAs, or (ii) inorganic elongated elements such as carbon nanotubes, graphene nanoribbons, semiconductor nanowires/nanoribbons (such as Si, InP, ZnO, GaAs), two dimensional chalcogenide semiconductor nanoribbons (such as MoS₂ or WS₂), or other semiconductor nanoscale elements.

In various embodiments, semiconductor nanoribbons or nanowires aligned by this method can have dimensions <30 nm or <20 nm, preferably <10 nm, and more preferably <6 nm in thickness, width or diameter. Single wall carbon nanotubes (SWCNTs) can have a diameter typically in the range of 0.8-1.3 nm. Non-semiconductor metallic nanowires such as Ag nanowires, or ceramic nanowires such as TiO₂ or Al₂O₃ (e.g., having <20 nm, or <10 nm diameter) can also be aligned by this method, such as for the purpose of using the aligned nanowires or aligned DNAs/peptides as a shadow mask for RIE or oxygen plasma etch to form nanoribbons of graphene or MoS₂.

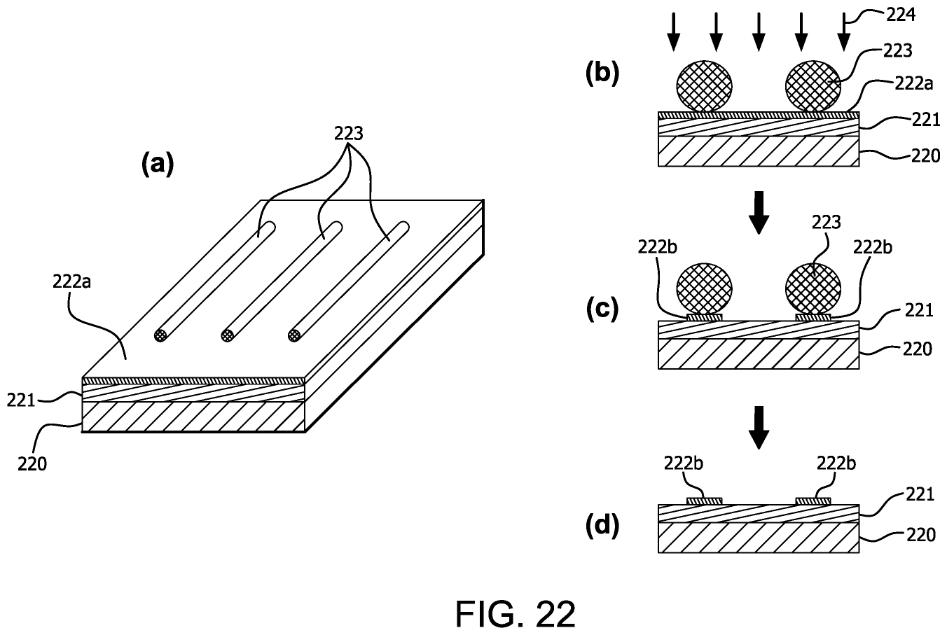

FIG. 22 illustrates a method of using pre-aligned nanowires or nanoribbons (e.g., obtained by the methods of FIGS. 19-21) as a shadow mask that can be later removed. As shown in FIG. 22(a), various aspects of the method begin with a structure having shear-aligned and parallel nanowires or nanoribbons 223 disposed on a graphene or MoS₂ layer 222a disposed on an underlying substrate comprising a layer of SiO₂ 221 on a layer of Si 220. The shear-aligned nanoribbons or nanowires 223 for this method may include, but are not limited to, oligonucleotides such as DNA, polypeptides, their associated assemblies, AgNW, CNTs, ZnO, InP, SiNW, and so forth.

With reference now to FIGS. 22(b) and (c), RIE or plasma etch 224 is used to remove only the exposed portions of the graphene or MoS₂ layer 222a, to leave behind only the portions of the graphene or MoS₂ layer 222b residing underneath each of the nanowires or nanoribbons 223. The last step of the method is illustrated in FIGS. 22(c) and (d) where the nanowires or nanotubes 223 are dissolved or burned away, leaving behind only the graphene or MoS₂ portions 222b that were underneath the nanowires or nanoribbons. Removal of the nanowires or nanoribbons can be affected by burning or etching (e.g., if comprising DNA, polypeptides) by dissolving (e.g., if an Ag alloy) or by peeling away the nanowires or nanoribbons (e.g., if Al₂O₃) to expose graphene or MoS₂ structures that can be subsequently transferred. In this way, the shear-aligned nanowires or nanoribbons are used as a mask to pattern an underlying layer of graphene, MoS₂, or other planar semiconductor layers that can then be used as is or transferred to another structure.

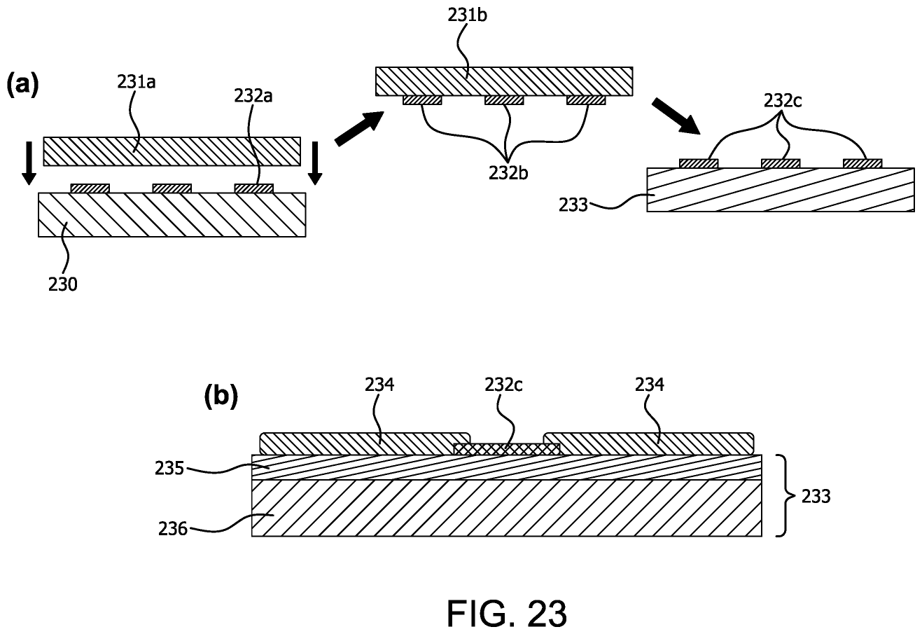

FIG. 23 illustrates various embodiments of a transfer method used to transfer a group of pre-aligned and parallel nanowires or nanoribbons from a substrate to a device structure. In various embodiments, a device structure comprises a surface for receiving the transferred parallel nanowires or nanoribbons, and the surface may comprise Si or SiO₂ over a silicon base substrate. After the parallel nanowires or nanoribbons are transferred onto the device structure, pairs of electrodes can then be deposited over the transferred parallel nanowires or nanoribbons to produce arrays of FETs. The pairs of electrodes are deposited in a parallel array wherein the separation between each pair of electrodes dimensionally match the separation between the nanowires or nanoribbons previously transferred. Each pair of electrodes comprise a first electrode and a second electrode spaced apart from the first electrode by a nanogap. In embodiments where the electrodes are later deposited over the previously transferred parallel nanowires or nanoribbons, the first and second electrodes in any one pair of electrodes encase the two ends of one nanowire or nanoribbon. Stated another way, the nanogap between a first and second electrode will be shorter than the length of a nanowire or nanoribbon such that each electrode is disposed over an end of the nanowire or nanoribbon and electrical connection between the first and second electrodes in each pair of electrodes is established.

As illustrated in FIG. 23(a), the method begins with a PDMS or adhesive stamp 231a used to pick up a group of pre-aligned and parallel nanowires or nanoribbons 232a from substrate 230. In various embodiments, the stamp 231a may comprise a material that can soften upon heating, such as a polymeric release tape, to release the pre-aligned and parallel nanowires or nanoribbons 232a from its surface. As mentioned above, the pre-aligned and parallel nanowires or nanoribbons may have been formed as a plurality of nanowires or nanoribbons on a stepped or grooved substrate, captured and aligned on a grooved substrate, or may have been shear-aligned on a substrate by a scraper being dragged through a liquid suspension of randomly oriented nanowires or nanoribbons.

With continued reference to FIG. 23(a), the stamp 231b has picked up the pre-aligned and parallel nanowires or nanoribbons 232b where they are transferred onto the device structure 233, appearing as pre-aligned and parallel nanowires or nanoribbons 232c on the device structure 233 once the stamp is softened and peeled off. Alternatively, an optional combination with a deposited metal film can be utilized for stronger pick-up of the aligned nanowires and nanoribbons for higher yield transfer.

With reference now to FIG. 23(b), pairs of electrodes 234 are deposited and patterned over the pre-aligned and transferred nanowires or nanoribbons 232c. Each pair of electrodes thus deposited comprise a first electrode and a second electrode spaced apart from the first electrode by a nanogap. In various embodiments, the device structure 233 comprises a dielectric substrate layer SiO₂ 235 disposed over a Si base layer 236. FIG. 23(b) illustrates a cross-sectional view of just one device structure in an array of structures produced by the nanowire or nanoribbon stamp transfer process and subsequent electrode depositing and patterning. Of note is that the method depicted in FIG. 23 provides the device structure (b) wherein each pair of electrodes 234, and each nanowire or nanoribbon 232c connecting the two electrodes 234 in each pair of electrodes, reside on a common substrate surface 235, because the electrodes were deposited after transfer of the nanowires or nanoribbons to the device structure 233.

In various embodiments of the method depicted in FIG. 23, physical and electrical bonding of the stamp-transferred nanowires or nanoribbons onto the electrode surface can be accomplished by van der Waals forces, or alternatively and optionally, a dielectric or metallic anchor coating on the end regions of the nanowire or nanoribbon can utilized for a more robust and secured bridge array. For enhanced adhesion of the transferred nanowires or nanoribbons onto electrodes, a Ti, Cr, Zr or other type of adhesion layer may be utilized.

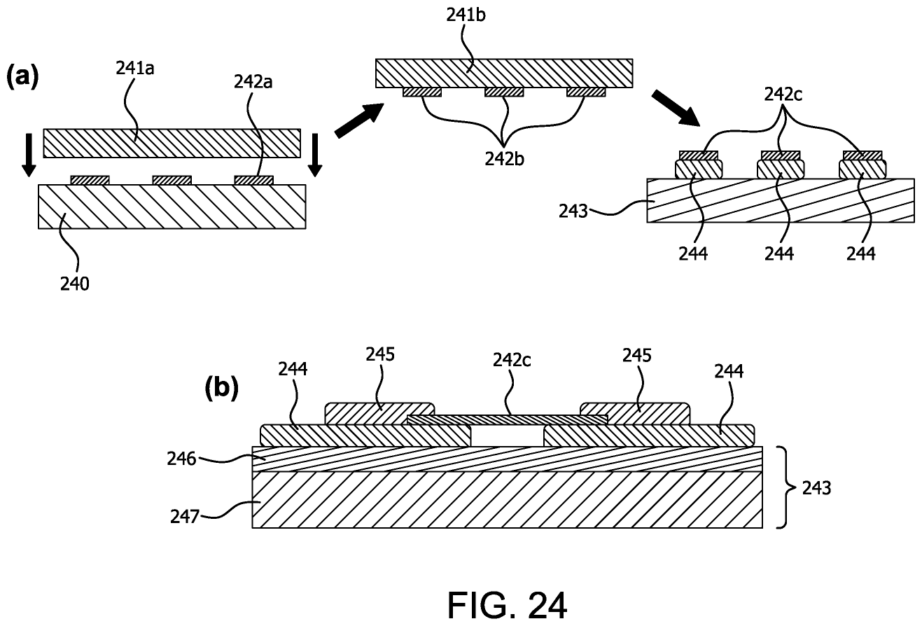

FIG. 24 depicts an alternative transfer method that produces bridging nanowires or nanoribbons rather than a structure wherein the nanowires or nanoribbons are on the same device structure surface as the electrodes. In this method, a stamp 241a is used to pick up a group of pre-aligned and parallel nanowires or nanoribbons 242a from a substrate 240, where they reside as a plurality, and transfer the group to a surface of a device structure 243 that already includes electrodes 244 arranged in pairs on the device surface. Each pair of electrodes comprises a first electrode and a second electrode spaced-apart from the first electrode by a nanogap. The transfer results in an array of FET structures where each nanowire or nanoribbon 242c is on top of a pair of electrodes 244 on the device structure 243. As shown in FIG. 24(b), the resulting structure comprises pairs of electrodes 244 on the device structure 243 and a nanowire or nanoribbon connecting the two electrodes in each pair of electrodes and bridging over the nanogap between electrodes. In various aspects of the method, a device structure is prepared with an array of pairs of electrodes, and then the stamp transfer process moves the group of nanowires or nanoribbons on top of electrodes pairs such that a single nanowire or nanoribbon is positioned over the top of only one electrode pair. The spacing between nanowires or nanoribbons is matched to the spacing between the pairs of electrodes such that only one nanowire or nanoribbon bridges only one pair of electrodes. In various aspects of the method, a dielectric (e.g., resin or silica) or metallic layer 245 may be used as an anchor coating on the ends of the nanowire or nanoribbon 242c. In various embodiments, the device structure 243 comprises a SiO₂ layer 246, onto which the electrodes 244 are disposed, on top of an underlying Si base layer 247.

In various embodiments, the physical attachment and strong electrical connection of the nanowire or nanoribbon onto the pre-existing array of electrode surfaces (such as made of Au, Ag, Pt, Pd, Ru, Rh, Al, Cu, Ni, or alloys, with an optional adhesion layer of Ti, Cr, Zr, etc.) can comprise van der Waals forces.

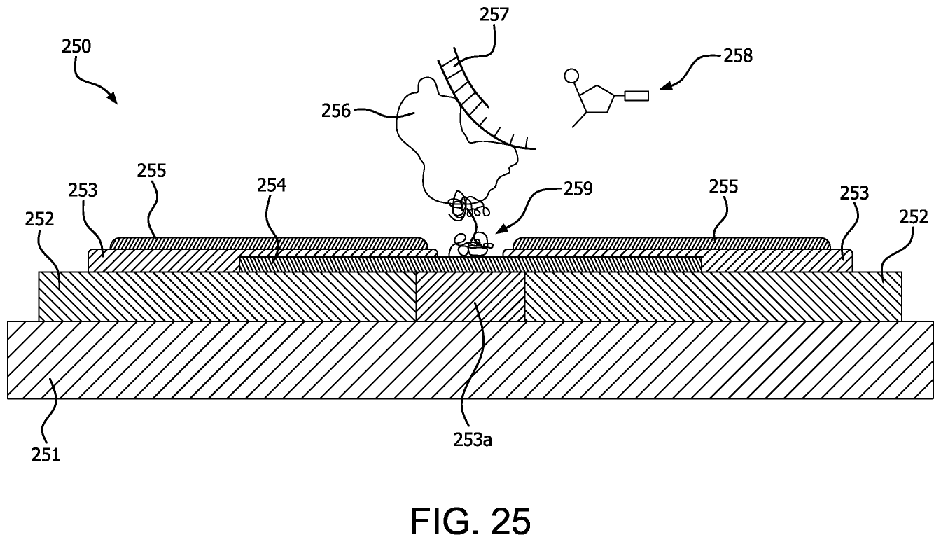

FIG. 25 illustrates a cross-sectional view of a completed single-molecule sensor 250 that comprises part of a large array of sensors manufacturable by the various methods described herein. The completed sensor 250 comprises a substrate 251 that may comprise SiO₂ on Si, a pair of electrodes 252 that are spaced-apart to form a nanogap 253a. The nanogap 253a may be open (not filled with any material) or may be filled in with semiconductor material. The sensor 250 further comprises a bridging semiconductor layer 254 in the form of a nanowire or nanoribbon. Each of the bridging nanowire or nanoribbon layers 254 can be formed in parallel arrays by the methods herein and transferred on top of the electrodes 252. Each device 250 may optionally include an anchoring dielectric or metal layer 253 to secure the bridging nanowire or nanoribbon layer 254 onto an across each pair of electrodes 252. Further, each device 250 may optionally include a blocking layer 255 to limit an exposed region of the bridging nanowire or nanoribbon layer 254. This aspect was described above, where a region of about 5 nm was mentioned as preferred so that only one molecule can bind to the exposed nanowire or nanoribbon layer 254. In various embodiments, the blocking layer 255 may comprise PEG or Teflon or some other agent that shields biomolecule adhesion. Each of the devices include a single enzyme molecule 256, such as a DNA or RNA polymerase, bonded to the exposed bridging nanowire or nanoribbon layer 254 via a streptavidin-biotin linkage 259 or another suitable linker.

As depicted in FIG. 25, each device 250 in an array of devices operates by sensing interactions of various nucleotides or modified nucleotides 258 with the enzyme molecule 256, such as when the enzyme is processing a DNA or RNA template 257 attached thereon. The interactions of the nucleotides 258 with enzyme 256 during sequencing are detected as perturbations in an electrical parameter in the device, such as current, voltage or impedance. In various configurations, a gate electrode may be wired in below the nanogap 253a, such as comprising a buried gate.

In various embodiments, massively parallel electronic sequencing analysis can be performed with many devices 250 organized into a system, having as many as 10,000 or even at least 1 million devices 250.

### ADDITIONAL CONSIDERATIONS

In various embodiments, a single-molecule biosensor comprises: a conducting electrode pair disposed on a substrate, the electrode pair comprising a first electrode and a second electrode spaced-apart from the first electrode by a nanogap; a contiguous transition metal dichalcogenide (TMD) layer disposed on the first electrode and on the second electrode, the TMD layer configured as a bridge suspended over the nanogap; an enzyme molecule attached to a region of the TMD layer suspended over the nanogap; and a microfluidic system encasing the conducting electrode pair, the contiguous TMD layer and the enzyme molecule attached thereto.

In various embodiments, the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, and mixtures thereof.

In various embodiments, the TMD layer comprises a TMD selected from the group consisting of MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, and mixtures thereof.

In various embodiments, the TMD layer comprises at least one TMD of structure MSe₍₂₋ₓ₎ or MSe₍₂₊ₓ₎, wherein x is 0-0.3, and M is Mo, W, Ti, Zr, Hf, V, Nb, Ta, Tc, Re, Co, Rh, Ir, Ni, Pd, or Pt.

In various embodiments, the TMD layer comprises a TMD selected from the group consisting of MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

In various embodiments, the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

In various embodiments, the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

In various embodiments, the substrate comprises SiO₂ or Al₂O₃.

In various embodiments, the pair of conducting electrodes comprise at least one of Au, Pt, Ag, Pd, Rh, Ru, or alloys thereof.

In various embodiments, the nanogap is from about 1 nm to about 50 nm.

In various embodiments, the single-molecule biosensor further comprises a dielectric, ceramic or polymer coating layer disposed on the TMD layer on a side opposite the first and second electrodes, wherein the dielectric, ceramic or polymer coating layer includes an opening on the region of the TMD layer suspended over the nanogap, the opening leaving an exposed portion of the TMD layer therein, the opening dimensioned to accommodate only one enzyme molecule.

In various embodiments, the dielectric, ceramic or polymer layer comprises PMMA or SiO₂.

In various embodiments, the enzyme molecule comprises a DNA polymerase or an RNA polymerase.

In various embodiments, the enzyme molecule is attached to the TMD layer via a biotin-streptavidin linkage.

In various embodiments, the TMD layer includes as least one of vacancy defects, interstitial defects, and aggregated defects.

In various embodiments of the present disclosure, a DNA or RNA sequencing device comprises: an electrode array of conducting electrode pairs disposed on a substrate, each pair of electrodes comprising a source electrode and a drain electrode spaced-apart from the source electrode by a nanogap; a contiguous transition metal dichalcogenide (TMD) layer disposed on the first electrode and on the second electrode, the TMD layer configured as a bridge suspended over the nanogap; a polymerase enzyme molecule attached to a region of the TMD layer over the nanogap; and a microfluidic system encasing the conducting electrode pair, the contiguous TMD layer and the enzyme molecule attached thereto.

In various embodiments, the TMD layer comprises at least one TMD of structure MX₍₂₋ₓ₎ or MX₍₂₊ₓ₎, wherein X is S, Se or Te; x is 0-0.3; and M is Mo, W, Ti, Zr, Hf, V, Nb, Ta, Tc, Re, Co, Rh, Ir, Ni, Pd, or Pt.

In various embodiments, a single-molecule biosensor comprises: a conducting electrode pair disposed on a substrate, the electrode pair comprising a first electrode and a second electrode spaced-apart from the first electrode by a nanogap; a contiguous Si-material layer disposed on the first electrode and on the second electrode, the Si-material layer configured as a bridge suspended over the nanogap; an enzyme molecule attached to a region of the Si-material layer suspended over the nanogap; and a microfluidic system encasing the conducting electrode pair, the contiguous Si-material layer and the enzyme molecule attached thereto.

In various embodiments, the Si-material layer comprises a crystalline silicon, a p-type doped silicon semiconductor material or an n-type doped silicon semiconductor material.

In various embodiments, the substrate comprises SiO₂ or Al₂O₃.

In various embodiments, the pair of conducting electrodes comprise at least one of Au, Pt, Ag, Pd, Rh, Ru, or alloys thereof.

In various embodiments, the nanogap is from about 1 nm to about 50 nm.

In various embodiments, the single-molecule biosensor further comprises a dielectric, ceramic or polymer coating layer disposed on the Si-material layer on a side opposite the first and second electrodes, wherein the dielectric, ceramic or polymer coating layer includes an opening on the region of the Si-material layer suspended over the nanogap, the opening leaving an exposed portion of the Si-material layer therein, the opening dimensioned to accommodate only one enzyme molecule.

In various embodiments, the dielectric, ceramic or polymer layer comprises PMMA or SiO₂.

In various embodiments, the enzyme molecule comprises a DNA polymerase or an RNA polymerase.

In various embodiments, the enzyme molecule is attached to the Si-material layer via a biotin-streptavidin linkage.

In various embodiments, the Si-material layer was obtained from a silicon-on-insulator (SOI) wafer.

In various embodiments, the substrate and Si-Material layer comprise a silicon-on-insulator (SOI) wafer.

In various embodiments of the present disclosure, a DNA or RNA sequencing device comprises: an electrode array of conducting electrode pairs disposed on a substrate, each pair of electrodes comprising a source electrode and a drain electrode spaced-apart from the source electrode by a nanogap; a contiguous Si-material layer disposed on the first electrode and on the second electrode, the Si-material layer configured as a bridge suspended over the nanogap; a polymerase enzyme molecule attached to a region of the Si-material layer over the nanogap; and a microfluidic system encasing the conducting electrode pair, the contiguous Si-material layer and the enzyme molecule attached thereto.

In various embodiments of the present disclosure, a single-molecule biosensor comprises: a conducting electrode pair disposed on a substrate, the electrode pair comprising a first electrode and a second electrode spaced-apart from the first electrode by a nanogap of fixed width; a TMD, Si, or doped Si-semiconductor layer disposed on the substrate electrically connecting the first and second electrodes; a dielectric layer disposed completely over the first and second electrodes to encase the first and second electrodes, and disposed partially over the TMD, Si, or doped Si-semiconductor layer so as to leave an exposed portion of the TMD, Si, or doped Si-semiconductor layer having a width less than the width of the nanogap; an enzyme molecule attached to the exposed portion of the TMD, Si, or doped Si-semiconductor layer; and a microfluidic system encasing the conducting electrode pair, the contiguous TMD layer and the enzyme molecule attached thereto.

In various embodiments, the TMD layer comprises at least one TMD having a structure MS₍₂₋ₓ₎, MS₍₂₊ₓ₎, MSe₍₂₋ₓ₎, MSe₍₂₊ₓ₎, MTe₍₂₋ₓ₎ or MTe₍₂₊ₓ₎, wherein x is 0-0.3, and M is Mo, W, Ti, Zr, Hf, V, Nb, Ta, Tc, Re, Co, Rh, Ir, Ni, Pd, or Pt.

In various embodiments, the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

In various embodiments, the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

In various embodiments, the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

In various embodiments, the substrate comprises Si, SiO₂ on Si, or Al₂O₃ on Si.

In various embodiments, the pair of conducting electrodes comprise at least one of Au, Ag, Pd, Pt, Ru, Rh, Al, Cu, Ni, or alloys thereof.

In various embodiments, the Si, or doped Si-semiconductor layer comprises single crystalline silicon, n-type doped silicon, or p-type doped silicon.

In various embodiments, the width of the nanogap is from about 20 nm to about 100 nm, and wherein the width of the exposed portion of the TMD, Si, or doped Si-semiconductor layer is about 5 nm.

In various embodiments, the enzyme molecule comprises a DNA polymerase or an RNA polymerase.

In various embodiments of the present disclosure, a DNA or RNA sequencing device comprises: a pair of electrodes disposed on a substrate, the pair comprising a first electrode and a second electrode spaced-apart from the first electrode by a nanogap having a width W1; a first nanopillar electrically attached to the first electrode and a second nanopillar electrically attached to the second electrode, wherein the two nanopillars are separated by a width W2 and wherein W2≥W1; a dielectric layer disposed over the electrode pair and over the nanogap to surround the nanopillars such that only a top surface of each nanopillar is exposed; a TMD, Si, or doped Si-semiconductor layer disposed on the dielectric layer and over the exposed top surface of each nanopillar, electrically connecting the first and second nanopillars; an enzyme molecule attached to a region of the TMD, Si, or doped Si-semiconductor layer between the nanopillars and directly over the nanogap; and a microfluidic system encasing the electrode pair, the TMD, Si, or doped Si-semiconductor layer and the enzyme molecule attached thereto.

In various embodiments, the TMD layer comprises at least one TMD having a structure MS₍₂₋ₓ₎, MS₍₂₊ₓ₎, MSe₍₂₋ₓ₎, MSe_{(2+X)}, MTe₍₂₋ₓ₎ or MTe₍₂₊ₓ₎, wherein x is 0-0.3, and M is Mo, W, Ti, Zr, Hf, V, Nb, Ta, Tc, Re, Co, Rh, Ir, Ni, Pd, or Pt.

In various embodiments, the TMD layer comprises a TMD selected from the group consisting of MoS₂, WS₂, TiS₂, ZrS₂, HfS₂, VS₂, NbS₂, TaS₂, TcS₂, ReS₂, CoS₂, RhS₂, IrS₂, NiS₂, PdS₂, PtS₂, MoSe₂, WSe₂, TiSe₂, ZrSe₂, HfSe₂, VSe₂, NbSe₂, TaSe₂, TcSe₂, ReSe₂, CoSe₂, RhSe₂, IrSe₂, NiSe₂, PdSe₂, PtSe₂, MoTe₂, WTe₂, TiTe₂, ZrTe₂, HfTe₂, VTe₂, NbTe₂, TaTe₂, TcTe₂, ReTe₂, CoTe₂, RhTe₂, IrTe₂, NiTe₂, PdTe₂, PtTe₂, and mixtures thereof.

In various embodiments, the TMD layer comprises a mixed TMD compound selected from the group consisting of Mo(SₓSe_{y}Te_{z})₂, W(SₓSe_{y}Te_{z})₂, Ti(SₓSe_{y}Te_{z})₂, Zr(SₓSe_{y}Te_{z})₂, Hf(SₓSe_{y}Te_{z})₂, V(SₓSe_{y}Te_{z})₂, Nb(SₓSe_{y}Te_{z})₂, Ta(SₓSe_{y}Te_{z})₂, Tc(SₓSe_{y}Te_{z})₂, Re(SₓSe_{y}Te_{z})₂, Co(SₓSe_{y}Te_{z})₂, Rh(SₓSe_{y}Te_{z})₂, Ir(SₓSe_{y}Te_{z})₂, Ni(SₓSe_{y}Te_{z})₂, Pd(SₓSe_{y}Te_{z})₂, Pt(SₓSe_{y}Te_{z})₂, and mixtures thereof, wherein (x+y+z) is 0.7-1.3.

In various embodiments, the TMD layer comprises at least one TMD compound of structure M_{(1-w)}N_{y}X_{(2-z)}Y_{z}, wherein M is Al, Si, Ga, Ge, In, Sn, Sb, Bi, Na, K, Ca, Mg, Sr, or Ba; X is S, Se, or Te; Y is Li, B, C, N, O, P, F, Cl, or I; w is 0-0.3; and z is 0-0.3.

In various embodiments, the dielectric layer comprises PMMA, SiO₂ or HSQ.

In various embodiments, the first and second electrodes comprise at least one of Au, Ag, Pd, Pt, Ru, Rh, Al, Cu, Ni, or alloys thereof, and wherein the first and second nanopillars comprise Au, Pd, Pt, Ru, or Rh.

In various embodiments, the Si, or doped Si-semiconductor layer comprises single crystalline silicon, n-type doped silicon, or p-type doped silicon.

In various embodiments, W1 is from about 5 nm to about 20 nm, and W2 is from about 5 nm to about 100 nm.

In various embodiments, the enzyme molecule comprises a DNA polymerase or an RNA polymerase.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A method of manufacturing a sensor device, the method comprising:
forming a plurality of parallel aligned nanowires or nanoribbons on a substrate;
providing a device structure comprising pairs of electrodes disposed in a parallel array on a surface of the device structure, each pair of electrodes in the array comprising a first electrode and a second electrode spaced apart from the first electrode by a nanogap;
transferring a group of the parallel aligned nanowires or nanoribbons from the substrate onto the array of pairs of electrodes such one nanowire or nanoribbon electrically connects the first and second electrodes in each pair of electrodes and forms a bridge suspended over the nanogap of each electrode pair;
patterning a dielectric layer over the parallel aligned nanowires or nanoribbons so as to leave openings in the dielectric layer, wherein each opening exposes a single region of nanowire or nanoribbon disposed over each nanogap in each pair of electrodes; and
attaching a molecule to each exposed region of nanowire or nanoribbon.

2. The method of claim 1, wherein the molecule comprises a DNA polymerase enzyme or an RNA polymerase enzyme.

3. The method of claim 1, wherein the forming comprises growing the nanowires or nanoribbons on parallel steps or within parallel grooves configured in the substrate.

4. The method of claim 3, wherein the forming comprises growing nanowires in the parallel grooves, wherein the nanowires have a diameter of less than about 10 nm.

5. The method of claim 3, wherein the forming comprises growing nanoribbons on the parallel steps, wherein the nanoribbons have a width of less than about 10 nm.

6. The method of claim 1, wherein the forming comprises shear-aligning randomly oriented nanowires or nanoribbons in a liquid suspension on the substrate by dragging an edge of a scraper through the liquid suspension.

7. The method of claim 1, wherein the nanowires or nanoribbons comprise two dimensional transition metal chalcogenide (TMD) semiconductor nanoribbons, carbon nanotubes, graphene nanoribbons, silicon nanoribbons, n-type doped silicon semiconductor nanoribbons, or p-type doped silicon semiconductor nanoribbons.

8. The method of claim 1, wherein the surface of the device structure comprises Si, SiO₂ on Si, or Al₂O₃ on Si.

9. The method of claim 1, wherein the first and second electrodes in each pair of electrodes comprise at least one of Au, Ag, Pd, Pt, Ru, Rh, Al, Cu, Ni, or alloys thereof.

10. The method of claim 1, wherein the openings in the dielectric layer are each sized to less than about 30 nm equivalent diameter.

11. The method of claim 10, wherein the openings are circular, each having a diameter of less than about 10 nm.

12. A method of manufacturing a sensor device, the method comprising:
forming a plurality of parallel aligned nanowires or nanoribbons on a substrate;
transferring a group of the parallel aligned nanowires or nanoribbons from the substrate onto a surface of a device structure;
disposing an array of pairs of electrodes on the surface in parallel such that each pair of electrodes electrically connects to one nanowire or nanoribbon, wherein each pair of electrodes in the array comprises a first electrode and a second electrode spaced apart from the first electrode by a nanogap, and wherein one nanowire or nanoribbon electrically connects the first and second electrodes in each pair of electrodes;
patterning a dielectric layer over the parallel aligned nanowires or nanoribbons so as to leave one exposed region of nanowire or nanoribbon for each electrode pair, each exposed region positioned between the first and second electrodes in each pair of electrodes; and
attaching a single molecule to each exposed region of nanowire or nanoribbon.

13. The method of claim 12, wherein the molecule comprises a DNA polymerase enzyme or an RNA polymerase enzyme.

14. The method of claim 12, wherein the forming comprises growing the nanowires or nanoribbons on parallel steps or within parallel grooves configured in the substrate.

15. The method of claim 14, wherein the forming comprises growing nanowires in the parallel grooves, wherein the nanowires have a diameter of less than about 10 nm.

16. The method of claim 14, wherein the forming comprises growing nanoribbons on the parallel steps, wherein the nanoribbons have a width of less than about 10 nm.

17. The method of claim 12, wherein the forming comprises shear-aligning randomly oriented nanowires or nanoribbons in a liquid suspension on the substrate by dragging an edge of a scraper through the liquid suspension.

18. The method of claim 12, wherein the nanowires or nanoribbons comprise two dimensional transition metal chalcogenide (TMD) semiconductor nanoribbons, carbon nanotubes, graphene nanoribbons, silicon nanoribbons, n-type doped silicon semiconductor nanoribbons, or p-type doped silicon semiconductor nanoribbons.

19. The method of claim 12, wherein the surface of the device structure comprises Si, SiO₂ on Si, or Al₂O₃ on Si.

20. The method of claim 12, wherein the electrodes in each pair of electrodes comprise at least one of Au, Ag, Pd, Pt, Ru, Rh, Al, Cu, Ni, or alloys thereof.
